SEQUENCE LISTING (88 sequences)

SEQ ID NO: 1 - protein, 118 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSA

SEQ ID NO: 2 - protein, 10 aa, synthetic construct
GYTFTNYDIN

SEQ ID NO: 3 - protein, 20 aa, synthetic construct
WIGWIFPGDGSTQYNEKFKG

SEQ ID NO: 4 - protein, 9 aa, synthetic construct
QTTATWFAY

SEQ ID NO: 5 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 6 - protein, 11 aa, synthetic construct
RASQSISDYLH

SEQ ID NO: 7 - protein, 7 aa, synthetic construct
YASQSIS

SEQ ID NO: 8 - protein, 9 aa, synthetic construct
QNGHSFPLT

SEQ ID NO: 9 - protein, 117 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSS

SEQ ID NO: 10 - protein, 11 aa, synthetic construct
GYSITSGYTWH

SEQ ID NO: 11 - protein, 16 aa, synthetic construct
HIQYTGNTKYNPSLES

SEQ ID NO: 12 - protein, 8 aa, synthetic construct
GSTNSNDY

SEQ ID NO: 13 - protein, 113 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELK

SEQ ID NO: 14 - protein, 17 aa, synthetic construct
KSSQSLLYSNDQKNYLA

SEQ ID NO: 15 - protein, 7 aa, synthetic construct
WASTRES

SEQ ID NO: 16 - protein, 9 aa, synthetic construct
QQYFIYPLT

SEQ ID NO: 17 - protein, 118 aa, synthetic construct
QVQLVQSGAEVKKPGASVKVSCKASGYTFTSYWMHWVRQAPGQRLEWMGSIESNSAGTKYDERFKTRVTITRDTSASTAYMELSSLRSEDTAVYYCARFYYGNSFAYWGQGTLVTVSS

SEQ ID NO: 18 - protein, 10 aa, synthetic construct
GYTFTSYWMH

SEQ ID NO: 19 - protein, 17 aa, synthetic construct
SIESNSAGTKYDERFKT

SEQ ID NO: 20 - protein, 9 aa, synthetic construct
FYYGNSFAY

SEQ ID NO: 21 - protein, 113 aa, synthetic construct
DIVMTQSPSSLAVSLGERATINCKSSQSLLQSGNQKNYLTWYQQKPGQPPKLLIYWASTRESGVPDRFSGSGSGTDFTLTISSLQAEDVAVYYCQNNYIFPLTFGQGTKLEIK

SEQ ID NO: 22 - protein, 17 aa, synthetic construct
KSSQSLLQSGNQKNYLT

SEQ ID NO: 23 - protein, 7 aa, synthetic construct
WASTRES

SEQ ID NO: 24 - protein, 9 aa, synthetic construct
QNNYIFPLT

SEQ ID NO: 25 - protein, 118 aa, synthetic construct
QVQLVESGGGVVQPGRSLRLSCAVSGISLTRYGVHWVRQAPGKGLEWVGVIWAGGNTNYESALMSRFTISKDNSKNTVYLQMNSLRAEDTAMYYCGRAYGKGNSMDYWGQGTLVTVSS

SEQ ID NO: 26 - protein, 5 aa, synthetic construct
RYGVH

SEQ ID NO: 27 - protein, 16 aa, synthetic construct
VIWAGGNTNYESALMS

SEQ ID NO: 28 - protein, 10 aa, synthetic construct
AYGKGNSMDY

SEQ ID NO: 29 - protein, 113 aa, synthetic construct
EIVMTQSPPTLSLSPGERVTLSCKSSQSLLNSGNQKNYLTWYQQKPGQAPRLLIYWASTRESSIPARFSGSGSGTDFTLTISSLQPEDFAVYYCQNNYIYPLTFGGGTKLELK

SEQ ID NO: 30 - protein, 17 aa, synthetic construct
KSSQSLLNSGNQKNYLT

SEQ ID NO: 31 - protein, 7 aa, synthetic construct
WASTRES

SEQ ID NO: 32 - protein, 9 aa, synthetic construct
QNNYIYPLT

SEQ ID NO: 33 - protein, 118 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSS

SEQ ID NO: 34 - protein, 10 aa, synthetic construct
GYSITDYLLH

SEQ ID NO: 35 - protein, 17 aa, synthetic construct
WIDPEDAKTKYGQKFQS

SEQ ID NO: 36 - protein, 9 aa, synthetic construct
NSGTGTMDA

SEQ ID NO: 37 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 38 - protein, 11 aa, synthetic construct
KASQNVGSNVD

SEQ ID NO: 39 - protein, 7 aa, synthetic construct
KASIRYT

SEQ ID NO: 40 - protein, 9 aa, synthetic construct
MQSKSYPIT

SEQ ID NO: 41 - protein, 123 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 42 - protein, 10 aa, synthetic construct
GSRLDYYAVA

SEQ ID NO: 43 - protein, 17 aa, synthetic construct
CITYSGGSTNYAESVKG

SEQ ID NO: 44 - protein, 14 aa, synthetic construct
EPYGSSCPLSGYDT

SEQ ID NO: 45 - protein, 107 aa, synthetic construct [sequence omitted]

SEQ ID NO: 46 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 464 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 48 - protein, 347 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 49 - protein, 352 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 469 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 703 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 52 - protein, 448 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 703 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 586 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 55 - protein, 586 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 700 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 57 - protein, 583 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 58 - protein, 583 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 220 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 470 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 61 - protein, 475 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 353 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 63 - protein, 358 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSDIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 702 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 65 - protein, 447 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 702 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSEVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 585 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 68 - protein, 585 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSEVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 699 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 70 - protein, 582 aa, synthetic construct
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSSGGGGSGGGGSGGGGSEVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 220 aa, synthetic construct
DIVMTQSPSSLAVSLGERATINCKSSQSLLQSGNQKNYLTWYQQKPGQPPKLLIYWASTRESGVPDRFSGSGSGTDFTLTISSLQAEDVAVYYCQNNYIFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 220 aa, synthetic construct
EIVMTQSPPTLSLSPGERVTLSCKSSQSLLNSGNQKNYLTWYQQKPGQAPRLLIYWASTRESSIPARFSGSGSGTDFTLTISSLQPEDFAVYYCQNNYIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 470 aa, synthetic construct
DIVMTQSPSSLAVSLGERATINCKSSQSLLQSGNQKNYLTWYQQKPGQPPKLLIYWASTRESGVPDRFSGSGSGTDFTLTISSLQAEDVAVYYCQNNYIFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 74 - protein, 470 aa, synthetic construct
EIVMTQSPPTLSLSPGERVTLSCKSSQSLLNSGNQKNYLTWYQQKPGQAPRLLIYWASTRESSIPARFSGSGSGTDFTLTISSLQPEDFAVYYCQNNYIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 75 - protein, 475 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSDIVMTQSPSSLAVSLGERATINCKSSQSLLQSGNQKNYLTWYQQKPGQPPKLLIYWASTRESGVPDRFSGSGSGTDFTLTISSLQAEDVAVYYCQNNYIFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 475 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSEIVMTQSPPTLSLSPGERVTLSCKSSQSLLNSGNQKNYLTWYQQKPGQAPRLLIYWASTRESSIPARFSGSGSGTDFTLTISSLQPEDFAVYYCQNNYIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 703 aa, synthetic construct
QVQLVQSGAEVKKPGASVKVSCKASGYTFTSYWMHWVRQAPGQRLEWMGSIESNSAGTKYDERFKTRVTITRDTSASTAYMELSSLRSEDTAVYYCARFYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 78 - protein, 703 aa, synthetic construct
QVQLVESGGGVVQPGRSLRLSCAVSGISLTRYGVHWVRQAPGKGLEWVGVIWAGGNTNYESALMSRFTISKDNSKNTVYLQMNSLRAEDTAMYYCGRAYGKGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIK

SEQ ID NO: 79 - protein, 448 aa, synthetic construct
QVQLVQSGAEVKKPGASVKVSCKASGYTFTSYWMHWVRQAPGQRLEWMGSIESNSAGTKYDERFKTRVTITRDTSASTAYMELSSLRSEDTAVYYCARFYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 448 aa, synthetic construct
QVQLVESGGGVVQPGRSLRLSCAVSGISLTRYGVHWVRQAPGKGLEWVGVIWAGGNTNYESALMSRFTISKDNSKNTVYLQMNSLRAEDTAMYYCGRAYGKGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 703 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSQVQLVQSGAEVKKPGASVKVSCKASGYTFTSYWMHWVRQAPGQRLEWMGSIESNSAGTKYDERFKTRVTITRDTSASTAYMELSSLRSEDTAVYYCARFYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 703 aa, synthetic construct
EVQLVQSGAEVKKPGATVKISCKVSGYSITDYLLHWVQQAPGKCLEWMGWIDPEDAKTKYGQKFQSRVTITADTSTDTAYMELSSLRSEDTAVYYCAANSGTGTMDAWGQGTLVTVSSGGGGSGGGGSGGGGSDIVMTQSPATLSVSPGERATLSCKASQNVGSNVDWYQQKPGQSPRVLIYKASIRYTGIPARFSGSGSGTEFTLTISILQSEDFAVYFCMQSKSYPITFGCGTKLEIKGGGGSGGGGSGGGGSQVQLVESGGGVVQPGRSLRLSCAVSGISLTRYGVHWVRQAPGKGLEWVGVIWAGGNTNYESALMSRFTISKDNSKNTVYLQMNSLRAEDTAMYYCGRAYGKGNSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 118 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSS

SEQ ID NO: 84 - protein, 107 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIK

SEQ ID NO: 85 - protein, 330 aa, synthetic construct [sequence omitted]

SEQ ID NO: 86 - protein, 107 aa, synthetic construct [sequence omitted]

SEQ ID NO: 87 - protein, 214 aa, synthetic construct
DIVMTQSPATLSVSPGERATLSCKASQNVGSNVDWYQQKPGQSPRVLIYKASIRYTGIPARFSGSGSGTEFTLTISILQSEDFAVYFCMQSKSYPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 448 aa, synthetic construct
EVQLVQSGAEVKKPGATVKISCKVSGYSITDYLLHWVQQAPGKGLEWMGWIDPEDAKTKYGQKFQSRVTITADTSTDTAYMELSSLRSEDTAVYYCAANSGTGTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0055188 A1
(43) Pub. Date: Feb. 26, 2026

(54) BISPECIFIC ANTIBODY AND USE THEREOF

(71) Applicant: SHENGHE (CHINA) BIOPHARMACEUTICAL CO., LTD., Nanjing, Jiangsu (CN)

(72) Inventors: Jinhua ZHOU, Nanjing (CN); Chong ZHOU, Nanjing (CN); Chongbing WU, Nanjing (CN); Xiaoling JIANG, Nanjing (CN); Liusong YIN, Nanjing (CN); Huabing YANG, Nanjing (CN)

(73) Assignee: SHENGHE (CHINA) BIOPHARMACEUTICAL CO., LTD., Nanjing, Jiangsu (CN)

(21) Appl. No.: 19/104,761

(22) PCT Filed: Aug. 18, 2023
(86) PCT No.: PCT/CN2023/113765

§ 371 Date: Feb. 19, 2025

(30) Foreign Application Priority Data
Aug. 19, 2022 (WO) PCT/CN2022/113462

(51) Int. Cl. C07K 16/28

(52) U.S. Cl. CPC C07K 16/2827; C07K 16/28; C07K 16/2878; C07K 16/2896; C07K 2317/31; C07K 2317/52; C07K 2317/569; C07K 2317/622; C07K 2317/732; C07K 2317/734; C07K 2317/92

(57) ABSTRACT

A bispecific antibody, including: a first antibody or antigen-binding fragment thereof that specifically binds to a first antigen; and a second antibody or antigen-binding fragment thereof that specifically binds to a second antigen; where the first antigen is a tumor-associated antigen (TAA), and the second antigen is TNFR2. The bispecific antibody can target a tumor-associated antigen (TAA), and at the same time can stimulate the proliferation of CD8+ T cells, activate CD8+ T cells, and release anti-tumor factors such as IFNγ and IL2 by using a TNFR2 agonistic antibody; or can block TNF-TNFR2 and inhibit the proliferation of Treg by using a TNFR2 antagonistic antibody.

### TECHNICAL FIELD

The present invention belongs to the field of biomedicine, and specifically relates to a bispecific antibody that binds to a tumor-associated antigen (TAA) and TNFR2 and use thereof.

### BACKGROUND

Monoclonal antibody (mAb) has been widely used in the treatment of a variety of human diseases, including cancer, autoimmune diseases, infectious diseases, and cardiovascular diseases. Currently, more than 80 monoclonal antibodies, including mouse-derived, fully humanized, and chimeric antibodies, have been approved by the FDA for therapeutic use. Most of these antibodies are monospecific antibodies that recognize a single epitope and can be selected to activate or inhibit the activity of a target molecule through this single epitope. For example, trastuzumab, one of the best-selling anti-cancer protein therapeutics, blocks the growth of cancer cells by attaching itself to Her2 to prevent the attachment of human epidermal growth factor to Her2. Trastuzumab also stimulates the body's own immune cells to destroy the cancer cells. However, many physiological responses require cross-linking or co-joining of two or more different proteins or protein subunits to be triggered. In the case of activation of heteropolymeric cell-surface receptor complexes, for example, activation is usually achieved by interaction of the ligand with multiple structural domains on different proteins, resulting in proximity-associated activation of one or both receptor components.

Bispecific antibody (BsAb) is an antibody molecule that can bind two (or more) different antigenic epitopes at the same time. Compared with traditional monoclonal antibody, bispecific antibody has a unique mechanism of action: (1) Bispecific antibody can bind two or more different antigenic molecules or different epitopes of the same molecule at the same time, which is often not the case with combination drugs. (2) Mediation of intercellular interactions, bispecific antibodies can bind to two antigens on effector cells and target cells, bridging the gap between effector cells and target cells and promoting intercellular interactions, such as mediating the killing of tumor cells by immune cells. Therefore, bispecific antibodies have unique advantages that traditional monoclonal antibodies do not have.

Tumor necrosis factor receptor 2 (TNFR2), also known as tumor necrosis factor receptor superfamily 1B (TNFRSF1B) and CD120b, is a 75-KDA type I transmembrane protein that is a member of the tumor necrosis factor receptor superfamily, which contains an extracellular structural domain (ECD, residues 1-257) and an intracellular structural domain with a TRAF2-binding structural domain (ICD, residues 288-461). In normal T cells, TNFA-TNFR2 interactions trigger cell survival signals through the NFKB signaling pathway. However, in autoimmune T cells, TNFA-TNFR2 interaction triggers apoptotic signaling through the caspase pathway.

TNFR2 has now been demonstrated to enhance effector T cell (Teff) activation and reduce regulatory T cell (Treg)-mediated suppression. Activation of TNFR2 induces signaling through the mitogen-activated protein kinase (MAPK) signaling pathway, which coordinates the transcription of genes that promote evasion of apoptosis and cell proliferation through TRAF2/3 signaling and NFκB-mediated transcription. TNFR2 can be expressed not only on cancer cells, infiltrating tumor Treg, but also on effector Teff cells. Studies have shown that TNFR2 agonistic antibody can stimulate the proliferation of CD8+ T cells, activate CD8+ T cells, and release anti-tumor factors such as IFNγ and IL2; TNFR2 antagonistic antibody can block TNF-TNFR2 and inhibit the proliferation of Treg.

The present invention aims to develop a bispecific antibody that can specifically recognize a tumor-associated antigen, and at the same time can stimulate the proliferation of CD8+ T cells, activate CD8+ T cells, and release anti-tumor factors such as IFNγ and IL2 by using a TNFR2 agonistic antibody; or can block TNF-TNFR2 and inhibit the proliferation of Treg by using a TNFR2 antagonistic antibody.

### SUMMARY

In the present application, the inventors have developed a bispecific antibody with good performance capable of binding to tumor-associated antigen (TAA) and TNFR2. The bispecific antibody of the present invention can target a tumor-associated antigen (TAA), and at the same time can stimulate the proliferation of CD8+ T cells, activate CD8+ T cells, and release anti-tumor factors such as IFNγ and IL2 by using a TNFR2 agonistic antibody; or can block TNF-TNFR2 and inhibit the proliferation of Treg by using a TNFR2 antagonistic antibody.

The present invention provides a bispecific antibody, comprising: (a) a first antibody or antigen-binding fragment thereof that specifically binds to a first antigen; and (b) a second antibody or antigen-binding fragment thereof that specifically binds to a second antigen; wherein the first antigen is a tumor-associated antigen (TAA), and the second antigen is TNFR2.

In some embodiments, the first antibody or antigen-binding fragment thereof comprises a heavy chain and a light chain; and the second antibody or antigen-binding fragment thereof comprises scFv or VHH.

In some embodiments, the heavy chain variable region of one heavy chain and the light chain variable region of one light chain of the first antibody form an antigen-binding site, and the heavy chain variable region of another heavy chain and the light chain variable region of another light chain form an antigen-binding site.

In some embodiments, the bispecific antibody comprises one first antibody or antigen-binding fragment thereof and one or more scFv.

In some embodiments, the bispecific antibody comprises one first antibody or antigen-binding fragment thereof and one scFv, wherein the scFv is connected to the N-terminus of the heavy chain of the first antibody or antigen-binding fragment thereof.

In some embodiments, the bispecific antibody comprises one first antibody or antigen-binding fragment thereof and one scFv, wherein the scFv is connected to the C-terminus of the heavy chain of the first antibody or antigen-binding fragment thereof.

In some embodiments, the bispecific antibody comprises one first antibody or antigen-binding fragment thereof and two scFvs.

In some embodiments, the two scFvs are respectively connected to the N-terminus of two heavy chains of the first antibody or antigen-binding fragment thereof.

In some embodiments, the two scFvs are respectively connected to the C-terminus of two heavy chains of the first antibody or antigen-binding fragment thereof.

In some embodiments, the bispecific antibody comprises one first antibody that specifically binds to tumor associated antigen (TAA) and two scFvs that specifically bind TNFR2, wherein the two scFvs that specifically bind TNFR2 are connected to the N-terminus of each of two heavy chains of the first antibody.

In some embodiments, the bispecific antibody comprises one first antibody that specifically binds to tumor associated antigen (TAA) and two scFvs that specifically bind TNFR2, wherein the two scFvs that specifically bind TNFR2 are connected to the C-terminus of each of two heavy chains of the first antibody.

In some embodiments, the bispecific antibody comprises two first polypeptide chains and two second polypeptide chains, and for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the scFv.

In some embodiments, the bispecific antibody has two first polypeptide chains that are identical or different, and/or two second polypeptide chains that are identical or different.

In some embodiments, the bispecific antibody comprises one first antibody or antigen-binding fragment thereof and one or more VHH.

In some embodiments, the bispecific antibody comprises one first antibody or antigen-binding fragment thereof and one VHH, wherein the VHH is connected to the N-terminus of the heavy chain of the first antibody or antigen-binding fragment thereof.

In some embodiments, the bispecific antibody comprises one first antibody or antigen-binding fragment thereof and one VHH, wherein the VHH is connected to the C-terminus of the heavy chain of the first antibody or antigen-binding fragment thereof.

In some embodiments, the bispecific antibody comprises one first antibody or antigen-binding fragment thereof and two VHHs.

In some embodiments, the two VHHs are respectively connected to the N-terminus of two heavy chains of the first antibody or antigen-binding fragment thereof.

In some embodiments, the two VHHs are respectively connected to the C-terminus of two heavy chains of the first antibody or antigen-binding fragment thereof.

In some embodiments, the bispecific antibody comprises one first antibody that specifically binds to tumor associated antigen (TAA) and two VHHs that specifically bind TNFR2, wherein the two VHHs that specifically bind TNFR2 are connected to the N-terminus of each of two heavy chains of the first antibody.

In some embodiments, the bispecific antibody comprises one first antibody that specifically binds to tumor associated antigen (TAA) and two VHHs that specifically bind TNFR2, wherein the two VHHs that specifically bind TNFR2 are connected to the C-terminus of each of two heavy chains of the first antibody.

In some embodiments, the bispecific antibody comprises two first polypeptide chains and two second polypeptide chains, wherein, for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the VHH.

In some embodiments, the bispecific antibody has two first polypeptide chains that are identical or different, and/or two second polypeptide chains that are identical or different.

In some embodiments, the tumor-associated antigen (TAA) is selected from GPC3, CD19, CD20 (MS4A1), CD22, CD24, CD30, CD33, CD38, CD40, CD123, CD133, CD138, CDK4, CEA, Claudin18.2, AFP, ALK, B7H3, BAGE protein, BCMA, BIRC5 (survivin), BIRC7, β-catenin (β-catenin), brc-ab1, BRCA1, BORIS, CA9, CA125, carbonic anhydrase IX, cysteinyl asparagin-8 (caspase-8), CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, mesothelin, ML-IAP, MOv-γ, Muc1, Muc2, Muc3, Muc4, Muc5, Muc16, MUM1, Ras, RGS5, Rho, ROR1, SART-1, SART-3, STEAP1, STEAP2, TAG-72, TGF-β, TMPRSS2, Tang-Noble Antigen, TRP-1, TRP-2, tyrosinase and urolysin-3, 5T4. Preferably, the tumor-associated antigen (TAA) is selected from B7H3, CD24, or Claudin18.2.

In some embodiments, the heavy chain variable region of the scFv is connected to the light chain variable region by a linker L1.

In some embodiments, the scFv is connected to the N-terminusor C-terminusend of the heavy chain of the first antibody or antigen-binding fragment thereof by a linker L2.

In some embodiments, the VHH is connected to the N-terminusor C-terminusend of the heavy chain of the first antibody or antigen-binding fragment thereof by a linker L2.

In some embodiments, the linker L1 and linker L2 are identical or different. In some embodiments, the linker L1 and/or linker L2 has an amino acid sequence as shown in (G₄S)ₓ, where x is an integer selected from 1-6; preferably, the linker L1 and/or linker L2 is (G₄S)₂, (G₄S)₃ or (G₄S)₄.

In some embodiments, the heavy chain of the first antibody or antigen-binding fragment thereof comprises a heavy chain variable region and a heavy chain constant region, and the light chain comprises a light chain variable region and a light chain constant region; preferably, the first antibody or antigen-binding fragment thereof is a full-length antibody.

In some embodiments, the heavy chain of the first antibody or antigen-binding fragment thereof comprises a first Fc region and a second Fc region. In some embodiments, the first Fc region and the second Fc region are identical or different. In some embodiments, the first Fc region or the second Fc region is selected from IgG, IgA, IgD, IgE, IgM, or variants thereof. In some embodiments, the first Fc region or the second Fc region is selected from IgG1, IgG2, IgG3, IgG4, or variants thereof. In some embodiments, the first Fc region or the second Fc region comprises one or more amino acid mutations, preferably amino acid substitutions, insertions or deletions.

In some embodiments, the first antibody or antigen-binding fragment thereof specifically binds to B7H3, wherein HCDR1 of the first antibody or antigen-binding fragment thereof is as shown in SEQ ID NO: 2, or is a sequence having at least 80% identity to SEQ ID NO: 2; HCDR2 is as shown in SEQ ID NO: 3, or is a sequence having at least 80% identity to SEQ ID NO: 3; HCDR3 is as shown in SEQ ID NO: 4, or is a sequence having at least 80% identity to SEQ ID NO: 4; LCDR1 is as shown in SEQ ID NO: 6, or is a sequence having at least 80% identity to SEQ ID NO: 6; LCDR2 is as shown in SEQ ID NO: 7, or is a sequence having at least 80% identity to SEQ ID NO: 7; and LCDR3 as shown in SEQ ID NO: 8, or a sequence having at least 80% identity to SEQ ID NO: 8.

In some embodiments, the first antibody or antigen-binding fragment thereof specifically binds to CD24, wherein HCDR1 of the first antibody or antigen-binding fragment thereof is as shown in SEQ ID NO: 10, or is a sequence having at least 80% identity to SEQ ID NO: 10; HCDR2 is as shown in SEQ ID NO: 11, or is a sequence having at least 80% identity to SEQ ID NO: 11; HCDR3 is as shown in SEQ ID NO: 12, or is a sequence having at least 80% identity to SEQ ID NO: 12; LCDR1 is as shown in SEQ ID NO: 14, or is a sequence having at least 80% identity to SEQ ID NO: 14; LCDR2 is as shown in SEQ ID NO: 15, or is a sequence having at least 80% identity to SEQ ID NO: 15; and LCDR3 is as shown in SEQ ID NO: 16, or is a sequence having at least 80% identity to SEQ ID NO: 16.

In some embodiments, the first antibody or antigen-binding fragment thereof specifically binds to Claudin 18.2, wherein HCDR1 of the first antibody or antigen-binding fragment thereof is as shown in SEQ ID NO: 18, or is a sequence having at least 80% identity to SEQ ID NO: 18; HCDR2 is as shown in SEQ ID NO: 19, or is a sequence having at least 80% identity to SEQ ID NO: 19; HCDR3 is as shown in SEQ ID NO: 20, or is a sequence having at least 80% identity to SEQ ID NO: 20; LCDR1 is as shown in SEQ ID NO: 22, or is a sequence having at least 80% identity to SEQ ID NO: 22; LCDR2 is as shown in SEQ ID NO: 23, or is a sequence having at least 80% identity to SEQ ID NO: 23; and LCDR3 is as shown in SEQ ID NO: 24, or is a sequence having at least 80% identity to SEQ ID NO: 24.

In some embodiments, the first antibody or antigen-binding fragment thereof specifically binds to Claudin 18.2, wherein HCDR1 of the first antibody or antigen-binding fragment thereof is as shown in SEQ ID NO: 26, or is a sequence having at least 80% identity to SEQ ID NO: 26; HCDR2 is as shown in SEQ ID NO: 27, or is a sequence having at least 80% identity to SEQ ID NO: 27; HCDR3 is as shown in SEQ ID NO: 28, or is a sequence having at least 80% identity to SEQ ID NO: 28; LCDR1 is as shown in SEQ ID NO: 30, or is a sequence having at least 80% identity to SEQ ID NO: 30; LCDR2 is as shown in SEQ ID NO: 31, or is a sequence having at least 80% identity to SEQ ID NO: 31; and LCDR3 is as shown in SEQ ID NO: 32, or is a sequence having at least 80% identity to SEQ ID NO: 32.

In some embodiments, the scFv specifically binds to TNFR2, and HCDR1 of the scFv is as shown in SEQ ID NO: 34, or is a sequence having at least 80% identity to SEQ ID NO: 34; HCDR2 is as shown in SEQ ID NO: 35, or is a sequence having at least 80% identity to SEQ ID NO: 35; HCDR3 is as shown in SEQ ID NO: 36, or is a sequence having at least 80% identity to SEQ ID NO: 36; and, LCDR1 is as shown in SEQ ID NO: 38, or is a sequence having at least 80% identity to SEQ ID NO: 38; LCDR2 is as shown in SEQ ID NO: 39, or is a sequence having at least 80% identity to SEQ ID NO: 39; and LCDR3 is as shown in SEQ ID NO: 40, or is a sequence having at least 80% identity to SEQ ID NO: 40.

In some embodiments, the VHH specifically binds to TNFR2, and HCDR1 of the VHH is as shown in SEQ ID NO: 42, or is a sequence having at least 80% identity to SEQ ID NO: 42; HCDR2 is as shown in SEQ ID NO: 43, or is a sequence having at least 80% identity to SEQ ID NO: 43; and HCDR3 is as shown in SEQ ID NO: 44, or is a sequence having at least 80% identity to SEQ ID NO: 44.

In some embodiments, the first antibody or antigen-binding fragment thereof specifically binds to B7H3, wherein the heavy chain variable region VH of the first antibody or antigen-binding fragment thereof is as shown in SEQ ID NO: 1, or is a sequence having at least 80% identity to SEQ ID NO: 1; and the light chain variable region VL is shown in SEQ ID NO: 5, or is a sequence having at least 80% identity to SEQ ID NO: 5.

In some embodiments, the first antibody or antigen-binding fragment thereof specifically binds to CD24, wherein the heavy chain variable region VH of the first antibody or antigen-binding fragment thereof is as shown in SEQ ID NO: 9, or is a sequence having at least 80% identity to SEQ ID NO: 9; and the light chain variable region VL is as shown in SEQ ID NO: 13, or is a sequence having at least 80% identity to SEQ ID NO: 13.

In some embodiments, the first antibody or antigen-binding fragment thereof specifically binds to Claudin 18.2, wherein the heavy chain variable region VH of the first antibody or antigen-binding fragment thereof is as shown in SEQ ID NO: 17, or is a sequence having at least 80% identity to SEQ ID NO: 17; and the light chain variable region VL is as shown in SEQ ID NO: 21, or is a sequence having at least 80% identity to SEQ ID NO: 21.

In some embodiments, the first antibody or antigen-binding fragment thereof specifically binds to Claudin 18.2, wherein the heavy chain variable region VH of the first antibody or antigen-binding fragment thereof is as shown in SEQ ID NO: 25, or is a sequence having at least 80% identity to SEQ ID NO: 25; and the light chain variable region VL is as shown in SEQ ID NO: 29, or is a sequence having at least 80% identity to SEQ ID NO: 29.

In some embodiments, the scFv specifically binds to TNFR2, and the heavy chain variable region VH of the scFv is as shown in SEQ ID NO: 33, or is a sequence having at least 80% identity to SEQ ID NO: 33; and the light chain variable region VL is as shown in SEQ ID NO: 37, or is a sequence having at least 80% identity to SEQ ID NO: 37.

In some embodiments, the VHH specifically binds to TNFR2, and the heavy chain variable region VH of the VHH is as shown in SEQ ID NO: 41 or is a sequence having at least 80% identity to SEQ ID NO: 41.

In some embodiments, the first polypeptide chain of the bispecific antibody is selected from the amino acid sequence of any one of SEQ ID NOs: 46-50, 59-63, 71-76, or is an amino acid sequence having at least 80% identity to the amino acid sequence of any one of SEQ ID NOs: 46-50, 59-63, 71-76; and the second polypeptide chain of the bispecific antibody is selected from the amino acid sequence of any one of SEQ ID NOs: 51-58, 64-70, 77-82, or is an amino acid sequence having at least 80% identity to the amino acid sequence of any one of SEQ ID NOs: 51-58, 64-70, 77-82.

In some embodiments, the bispecific antibody comprises:
(1) the first polypeptide chain as shown in SEQ ID NO: 46, and the second polypeptide chain as shown in SEQ ID NO: 51;
(2) the first polypeptide chain as shown in SEQ ID NO: 46, and the second polypeptide chain as shown in SEQ ID NO: 56;
(3) the first polypeptide chain as shown in SEQ ID NO: 46, and the second polypeptide chain as shown in SEQ ID NO: 53;
(4) the first polypeptide chain as shown in SEQ ID NO: 46, and the second polypeptide chain as shown in SEQ ID NO: 54;
(5) the first polypeptide chain as shown in SEQ ID NO: 46, and the second polypeptide chain as shown in SEQ ID NO: 57;
(6) the first polypeptide chain as shown in SEQ ID NO: 46, and the second polypeptide chain as shown in SEQ ID NO: 55;
(7) the first polypeptide chain as shown in SEQ ID NO: 46, and the second polypeptide chain as shown in SEQ ID NO: 58;
(8) the first polypeptide chain as shown in SEQ ID NO: 47, and the second polypeptide chain as shown in SEQ ID NO: 52;
(9) the first polypeptide chain as shown in SEQ ID NO: 50, and the second polypeptide chain as shown in SEQ ID NO: 52;
(10) the first polypeptide chain as shown in SEQ ID NO: 48, and the second polypeptide chain as shown in SEQ ID NO: 52;
(11) the first polypeptide chain as shown in SEQ ID NO: 49, and the second polypeptide chain as shown in SEQ ID NO: 52;
(12) the first polypeptide chain as shown in SEQ ID NO: 59, and the second polypeptide chain as shown in SEQ ID NO: 64;
(13) the first polypeptide chain as shown in SEQ ID NO: 59, and the second polypeptide chain as shown in SEQ ID NO: 69;
(14) the first polypeptide chain as shown in SEQ ID NO: 59, and the second polypeptide chain as shown in SEQ ID NO: 66;
(15) the first polypeptide chain as shown in SEQ ID NO: 59, and the second polypeptide chain as shown in SEQ ID NO: 67;
(16) the first polypeptide chain as shown in SEQ ID NO: 59, and the second polypeptide chain as shown in SEQ ID NO: 68;
(17) the first polypeptide chain as shown in SEQ ID NO: 59, and the second polypeptide chain as shown in SEQ ID NO: 70;
(18) the first polypeptide chain as shown in SEQ ID NO: 60, and the second polypeptide chain as shown in SEQ ID NO: 65;
(19) the first polypeptide chain as shown in SEQ ID NO: 61, and the second polypeptide chain as shown in SEQ ID NO: 65;
(20) the first polypeptide chain as shown in SEQ ID NO: 62, and the second polypeptide chain as shown in SEQ ID NO: 65;
(21) the first polypeptide chain as shown in SEQ ID NO: 63, and the second polypeptide chain as shown in SEQ ID NO: 65;
(22) the first polypeptide chain as shown in SEQ ID NO: 71, and the second polypeptide chain as shown in SEQ ID NO: 77;
(23) the first polypeptide chain as shown in SEQ ID NO: 72, and the second polypeptide chain as shown in SEQ ID NO: 78;
(24) the first polypeptide chain as shown in SEQ ID NO: 71, and the second polypeptide chain as shown in SEQ ID NO: 81;
(25) the first polypeptide chain as shown in SEQ ID NO: 72, and the second polypeptide chain as shown in SEQ ID NO: 82;
(26) the first polypeptide chain as shown in SEQ ID NO: 73, and the second polypeptide chain as shown in SEQ ID NO: 79;
(27) the first polypeptide chain as shown in SEQ ID NO: 74, and the second polypeptide chain as shown in SEQ ID NO: 80;
(28) the first polypeptide chain as shown in SEQ ID NO: 75, and the second polypeptide chain as shown in SEQ ID NO: 79; or
(29) the first polypeptide chain as shown in SEQ ID NO: 76, and the second polypeptide chain as shown in SEQ ID NO: 80.

The present invention provides an isolated nucleic acid molecule, comprising a nucleotide sequence encoding the bispecific antibody described in any of the foregoing. Preferably, the isolated nucleic acid molecule comprises a nucleotide sequence encoding the first polypeptide chain of the bispecific antibody described in any of the foregoing. Preferably, the isolated nucleic acid molecule comprises a nucleotide sequence encoding the second polypeptide chain of the bispecific antibody described in any of the foregoing.

The present invention provides a multifunctional fusion protein, comprising a bispecific antibody described in any of the foregoing.

In some embodiments, the multifunctional fusion protein further comprises one or more third antibody or antigen-binding portion thereof that bind specifically to other antigens. In some embodiments, the antigen that binds to the third antibody or antigen-binding portion thereof is selected from tumor-associated antigen (TAA) or immune checkpoint molecule. In some embodiments, the antigen that binds the third antibody or antigen-binding portion thereof is selected from GPC3, CD19, CD20 (MS4A1), CD22, CD24, CD30, CD33, CD38, CD40, CD123, CD133, CD138, CDK4, CEA, Claudin18.2, AFP, ALK, B7H3, BAGE proteins, BCMA, BIRC5 (survivin), BIRC7, β-catenin, brc-ab1, BRCA1, BORIS, CA9, CA125, carbonic anhydrase IX, cysteinyl asparagin-8 (caspase-8), CALR, CCR5, NA17, NKG2D, NY-BR1, NY-BR62, NY-BR85, NY ESO1, OX40, p15, p53, PAP, PAX3, PAX5, PCTA-1, PLAC1, PRLR, PRAME, PSMA (FOLH1), RAGE proteins, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, GM3, gp100, Her2, HLA/B-raf, HLA/k-ras, HLA/MAGE-A3, hTERT, IL13Rα2, LMP2, K-Light, LeY, MAGE-1, MAGE-2, MAGE-3, MAGE-4, MAGE-6, MAGE-12, MART-1, mesothelin, ML-IAP, MOv-γ, Muc1, Muc2, Muc3, Muc4, Muc5, Muc16, MUM1, Ras, RGS5, Rho, ROR1, SART-1, SART-3, STEAP1, STEAP2, TAG-72, TGF-β, TMPRSS2, Tow-No antigen, TRP-1, TRP-2, tyrosinase and urolysin-3, 5T4, PD-L1, CTLA4, PD-L2, PD-1, 4-1BB, CD47, TIGIT, GITR, TIM3, ILT4, TREM2, LAG3, CD27, or B7H4.

In some embodiments, the multifunctional fusion protein further comprises a cytokine. In some embodiments, the cytokine is selected from IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13Rα, IL-13 Rα2, IL-14, IL-15, IL-15Rα sushi, IL-16, IL-17, IL-18, IL-19, IL-20, IL-20R1, IL 20R2, IL-21, IL-21 Rα, IL-22, IL-23, IL-23R, IL-27R, IL-31R, TGF, VEGF, IFNγ, IFNα or GM-CSF.

In some embodiments, The present invention also provides use of the bispecific antibody described in any of the foregoing or the multifunctional fusion protein described in any of the foregoing in the preparation of a drug for the treatment of cancer. In some embodiments, the cancer is selected from astrocytoma of the human brain, human pharyngeal head carcinoma, adrenal gland tumors, AIDS-associated cancers, follicular soft tissue sarcoma, astrocytomas, bladder cancer, bone cancer, brain and spinal cord cancers, metastatic brain tumors, breast cancer, carotid body tumors, cervical cancer, chondrosarcoma, chordoma, smoky cell carcinoma of the kidney, clear cell carcinoma, colon cancer, colorectal cancer, connective tissue-promoting proliferative small round cell tumors, ventricular pleural cell tumor, Ewing tumor, extraosseous mucinous chondrosarcoma, osteofibrous dysplasia, osteofibrous dysplasia, gallbladder or bile duct cancer, gastric cancer, gestational trophoblastic disease, germ cell tumor, head and neck cancer, hepatocellular carcinoma, pancreatic islet cell tumor, Kaposi's sarcoma, renal carcinoma, leukemia, liposarcoma/malignant lipomatous neoplasms, hepatocellular carcinoma, lymphoma, lung carcinoma, neuronal ganglioblastic neoplasia, melanoma, meningioma, multiple endocrine neoplasia, multiple myeloma, myelodysplastic syndromes, adult neuroblastoma, neuroendocrine tumors, ovarian cancer, pancreatic cancer, papillary thyroid cancer, parathyroid adenoma, pediatric cancers, peripheral nerve sheath tumors, pheochromocytomas, pituitary tumors, prostate cancer, posterior uveal melanoma, metastatic renal carcinoma, rhabdomyosarcoma, rhabdomyosarcoma, sarcomas, skin cancers, soft tissue sarcomas, squamous cell carcinoma, synovial sarcoma, testicular cancer, thymic carcinoma, thymoma, metastatic thyroid cancer, or uterine cancer.

The present invention also provides use of the bispecific antibody described in any of the foregoing or the multifunctional fusion protein described in any of the foregoing in the preparation of a drug for the treatment of autoimmune disease. In some embodiments, the autoimmune disease is selected from graft-versus-host disease, rheumatoid arthritis, Crohn's disease, multiple sclerosis, colitis, psoriasis, autoimmune uveitis, aspergillosis, herpetic epidermolysis bullosa, or type I diabetes.

In some embodiments, the use is achieved by one or more of tumor immunotherapy, cell therapy, or gene therapy.

The present invention also provides a pharmaceutical composition, comprising a bispecific antibody described in any of the foregoing and a pharmaceutically acceptable carrier, diluent or excipient.

The present invention also provides a pharmaceutical composition, comprising a multifunctional fusion protein described in any of the foregoing and a pharmaceutically acceptable carrier, diluent or excipient.

The present invention also provides an antibody-drug conjugate, comprising a bispecific antibody described in any of the foregoing.

In some embodiments, the conjugate drug is selected from a cytotoxic agent, a small molecule chemical agent, or an immunotoxin.

### Abbreviations and Definitions of Terms

The following abbreviations are used herein. VH: antibody heavy chain variable region; VL: antibody light chain variable region; CDR: complementary determining region in the variable region of an immunoglobulin; FR: antibody framing region, i.e., amino acid residues in the variable region of an antibody other than CDR residues; IgG: immunoglobulin G.

The term “antibody” refers to a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells, or from animal immune serum, or from a phage library screen for reconstruction. Alternatively, they may be partially or completely synthesized by using techniques such as genetic recombination. Preferred antibody include, for example, antibody to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Within these isotypes, the antibody of the present invention may include IgG1, IgG2, IgG3 and/or IgG4.

The term “chimeric antibody” refers to an antibody made by fusing the variable region of a mouse-derived antibody with the constant region of a human antibody, which can reduce the immune response induced by the mouse-derived antibody. To create a chimeric antibody, a hybridoma secreting a mouse-specific monoclonal antibody is established, the variable region gene is cloned from the mouse hybridoma cells, the constant region gene of the human antibody is cloned as needed, the mouse variable region gene and the human constant region gene are connected to form a chimeric gene and then inserted into a human vector, and finally, the chimeric antibody molecule is expressed in the eukaryotic or prokaryotic industrial system.

Some of the antibodies used herein consist of an immunoglobulin molecule comprising two pairs of polypeptide chains, each pair having a light chain (LC) and a heavy chain (HC). Each heavy chain consists of a heavy chain variable region (VH) and a heavy chain constant region (CH). The heavy chain constant region consists of three structural domains (CH1, CH2 and CH3). Each light chain consists of a light chain variable region (VL) and a light chain constant region (CL), or only a light chain constant region (CL). The light chain constant region consists of a structural domain CL. The constant structural domain is not directly involved in antibody-antigen binding, but exhibits a variety of effector functions, such as the ability to mediate the binding of immunoglobulins to host tissues or factors, including various cells of the immune system (e.g., effector cells) and the first component of the classical complement system (Clq). The VH and VL regions can also be subdivided into regions with high variability (called complementary determining regions (CDRs)), with more conserved regions called framing regions (FRs) scattered in between. Each VH and VL consists of three CDRs and four FRs arranged from amino-terminal to carboxy-terminal in the following order: FR1, CDR1, FR2, CDR2, FR3, CDR3, FR4. The variable regions of each heavy chain/light chain pair (VH and VL) form the antigen-binding site, respectively.

The term “bispecific antibody” refers to a protein molecule that can specifically bind to two target antigens or target antigen epitopes. In the present invention, “bispecific antigen-binding protein” comprising an antibody or antigen-binding fragment (e.g., Fab, scFv, etc.) can be used interchangeably with “bispecific antibody” and “bis-antibody”.

The term “antigen-binding fragment” of an antibody refers to a polypeptide fragment of an antibody, such as a polypeptide fragment of a full-length antibody, which maintains the ability to specifically bind the same antigen bound by the full-length antibody and/or to compete with the full-length antibody for specific binding of the antigen, and which is also referred to as an “antigen-binding portion”. The antigen-binding portion of an antibody may be produced by recombinant DNA technology or by enzymatic or chemical breakage of the intact antibody. Non-limiting examples of antigen-binding fragments include Fab, Fab′, F(ab′)2, Fd, Fv, dAb and complementary determining region (CDR) fragments, single chain antibodies (e.g., scFv), chimeric antibodies, diabodies, linear antibodies, nanobodies (e.g., technology from Ablynx), structural domain antibodies (e.g., technology from Domantis), and such peptides comprising at least a portion of an antibody sufficient to confer specific antigen-binding ability to the peptide.

The term “antibody-drug conjugate” or “ADC” refers to a binding protein (e.g., an antibody or antigen-binding fragment thereof) that is coupled to one or more coupled drugs (which may optionally be therapeutic or cytotoxic agents), and whose structure typically consists of three parts: the antibody or antibody-like ligand, the drug portion, and a linker that couples the antibody or antibody-like ligand and the drug. ADCs generally have 1, 2, 3, 4, 5, 6, 7, 8, 9, or 10 drugs conjugated to the antibody.

The term “polypeptide” refers to a chain of amino acids of any length, irrespective of modification (e.g. phosphorylation or glycosylation). The term polypeptide includes proteins and fragments thereof. Polypeptides may be “exogenous”, meaning that they are “heterologous”, i.e., foreign to the host cell being utilized, e.g., human polypeptides produced by bacterial cells. Polypeptides are disclosed herein as sequences of amino acid residues. Those sequences are written from left to right in the direction from the amino terminus to the carboxy terminus. According to standard nomenclature, amino acid residue sequences are named in three-letter or one-letter codes.

The term “scFv” refers to a molecule comprising an antibody heavy chain variable structure domain (VH) and an antibody light chain variable structure domain (VL) linked by a linker. Such scFv molecules may have the general structure of NH2-VL-linker-VH-COOH or NH2-VH-linker-VL-COOH. Suitable prior art linkers comprise repeating GGGGS amino acid sequences or variants thereof, e.g., using 1-6 repeating GGGGS amino acid sequences or variants thereof.

The term “VHH” refers to a single antigen-binding polypeptide containing only a heavy chain variable region (VHH) derived from the variable structural domain of a heavy chain molecule that is naturally free of light chains, distinguishing it from the conventional VH of tetrameric immunoglobulins. Such VHH molecules can be derived from antibodies produced in species of camelid such as camels, American llamas, llamas, dromedary camels, alpacas and original camels. Species other than camelids can produce heavy chain molecules that naturally lack light chains, and such VHH is within the scope of the present application. The term “host cell” refers to a cell that has been or is capable of being transformed with a nucleic acid sequence and thus expressing a selected target gene. The term includes the progeny of a parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is attached. The term includes vectors that are self-replicating nucleic acid structures and vectors that are incorporated into the genome of the host cell into which they are introduced. Certain vectors are capable of directing the expression of nucleic acids operably connected to them. Such vectors are referred to herein as “expression vectors”.

The term “pharmaceutically acceptable carrier” includes any standard drug carrier, such as phosphate buffered saline solutions, water and emulsions, as well as various types of wetting agents.

The term “identity” is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA package.

The term “at least 80% identity” means that the percentage of amino acid residues in the candidate sequence that are identical to amino acid residues in the control polypeptide sequence is 80% or more, including 80%, 81%, 82%, 83%, 84%, 85%, 86%, 87%, 88%, 89%, 90%, 91%, 92%, 93%, 94%, 95%, 96%, 97%, 98%, 99%, 100%.

The term “specificity” refers to the fact that one of the molecules involved in specific binding does not show any significant binding to a molecule or molecules different from one or more of the binding partner molecules. In addition, the term is used when the structural domain containing the variable region of the antibody is specific to a particular epitope of a plurality of epitopes in the antigen. When the epitope to which the domain containing the antibody variable region binds is contained in several different antigens, an antigen-binding molecule comprising the domain containing the antibody variable region can bind a variety of antigens having said epitopes.

The term “tumor-associated antigen” or “TAA” refers to molecules (typically proteins, carbohydrates, lipids, or some combination thereof) that are expressed exclusively or in fragments on the surface of cancerous cells, and that can be used to preferentially target pharmacological agents to cancerous cells.

The term “epitope” refers to an antigenic determinant cluster in an antigen and the antigenic site bound by the structural domain of an antigen-binding molecule comprising an antibody variable region as disclosed in this specification. Thus, the epitope can be defined based on its structure. In addition, the epitope may also be defined based on the antigen-binding activity in the antigen-binding molecule that recognizes the epitope. When the antigen is a peptide or polypeptide, the epitope may be designated by the amino acid residues forming the epitope; when the epitope is a glycan chain, the epitope may be defined by its specific glycan chain structure.

The term “positive control” refers to a natural or engineered cell that binds or expresses a target protein, and a positive control herein refers to a single target positive control.

The term “isotype control” refers to a negative control that is used in the same experiment as the experimental sample of the same genus, same subtype, same dose, same immunoglobulin and immunoglobulin of subtype, same labeling, etc., and is used to eliminate the experimental background effect of the non-specifically bound sample on the experimental values in the experiment, and as a more illustrative control of the effect of the experiment.

The term “Treg” “Treg cells” or “regulatory T cells” is sometimes referred to as suppressor T cells, which are characterized by the expression of the biomarkers CD4, FOXP3, and CD25, and represent a subpopulation of T cells that regulate the immune system, maintain tolerance to self-antigens, and prevent autoimmune diseases. Treg are immunosuppressive and generally inhibit or downregulate the induction and proliferation of effector T (Teff) cells. Treg can develop in the thymus (so-called CD4+Foxp3+ “natural” Treg) or differentiate from naive CD4+ T cells in the periphery, e.g., after exposure to TGFβ or retinoic acid.

The term “Teff”, “Teff cell” or “effector T cell” refers to a cell that is formed by the proliferation and differentiation of T cells after they have been stimulated by an antigen. Effector T cells have the function of releasing lymphokines, and in this process, a small portion of T cells become memory T cells. Effector T cells contact with target cells and stimulate granule cytotoxicity, the released perforin through polymerization and the formation of small holes in the surface of the target cell, thus mediating the killing effect, and its target cell death process is similar to apoptosis. At the same time, effector T cells also release immunoreactive substances-lymphokines, such as interleukins and interferons.

There are a variety of methods/systems in the field to define and characterize CDRs that have been developed and refined over the years, including Kabat, Chothia, IMGT, AbM, and Contact; Kabat is the most commonly used and defines CDRs based on sequence variability; Chothia defines CDRs based on sequence variability based on the position of the cyclic region of the structure; the IMGT system defines CDRs based on sequence variability and position within the variable domain structure; AbM is defined based on Oxford Molecules' AbM antibody modeling software and is a compromise between Kabat and Chothia; Contact defines CDRs based on the analysis of complex crystal structures, and is similar to Chothia in several respects. Unless otherwise noted, Kabat is used herein to define CDRs. As a special note, some of the CDRs of this application are divided using defined criteria, such as HCDR1 shown in SEQ ID NO: 2, 10, 18, 34, 42, which is divided starting at the fourth position after the first cysteine of the heavy chain variable region, and the HCDR1 is generally 10-12 in length and cuts off at the amino acid one amino acid before the tryptophan.

### BRIEF DESCRIPTION OF THE DRAWINGS

FIG. 1 shows a depiction of exemplary bispecific antibody comprising a full-length antibody capable of specifically recognizing a tumor-associated antigen (TAA) and a scFv capable of specifically recognizing TNFR2, wherein the scFv is connected to the C-terminus of two heavy chains of the full-length antibody by a linker.

FIG. 2 shows a depiction of exemplary bispecific antibody comprising a full-length antibody capable of specifically recognizing a tumor-associated antigen (TAA) and a scFv capable of specifically recognizing TNFR2, wherein the scFv is connected to the N-terminus of two heavy chains of the full-length antibody by a linker.

FIG. 3 shows a depiction of exemplary bispecific antibody comprising a full-length antibody capable of specifically recognizing a tumor-associated antigen (TAA) and a VHH capable of specifically recognizing TNFR2, wherein the VHH is connected to the C-terminus of two heavy chains of the full-length antibody by a linker.

FIG. 4 shows a depiction of exemplary bispecific antibody comprising a full-length antibody capable of specifically recognizing a tumor-associated antigen (TAA) and a VHH capable of specifically recognizing TNFR2, wherein the VHH is connected to the N-terminus of two heavy chains of the full-length antibody by a linker.

FIG. 5 shows a depiction of exemplary bispecific antibody comprising a full-length antibody capable of specifically recognizing a tumor-associated antigen (TAA) and a scFv capable of specifically recognizing TNFR2, wherein the scFv is connected to the C-terminus of the two light chains of the full-length antibody by a linker.

FIG. 6 shows a depiction of exemplary bispecific antibody comprising a full-length antibody capable of specifically recognizing a tumor-associated antigen (TAA) and a scFv capable of specifically recognizing TNFR2, wherein the scFv is connected to the N-terminus of the two light chains of the full-length antibody by a linker.

FIG. 7 shows a depiction of exemplary bispecific antibody comprising a full-length antibody capable of specifically recognizing a tumor-associated antigen (TAA) and a VHH capable of specifically recognizing TNFR2, wherein the VHH is connected to the C-terminus of the two light chains of the full-length antibody by a linker.

FIG. 8 shows a depiction of exemplary bispecific antibody comprising a full-length antibody capable of specifically recognizing a tumor-associated antigen (TAA) and a VHH capable of specifically recognizing TNFR2, wherein the VHH is connected to the N terminus of the two light chains of the full-length antibody by a linker.

FIG. 9 shows the binding activity of antibodies TB-A1, TB-E, TB-B, and TB-F to TNFR2 proteins.

FIG. 10 shows the binding activity of antibodies TB-G, TB-D1, and TB-H to TNFR2 proteins.

FIG. 11 shows the binding activity of antibodies TB-A2 to TNFR2 proteins.

FIG. 12 shows the binding activity of antibodies TB-C1, TB-C2, and TB-D2 to TNFR2 proteins.

FIG. 13 shows the binding activity of antibodies TB-A1, TB-E, TB-B, and TB-F to B7H3 proteins.

FIG. 14 shows the binding activity of antibodies TB-G, TB-D1, and TB-H to B7H3 proteins.

FIG. 15 shows the binding activity of antibodies TB-A2, and TB-C1 to B7H3 proteins.

FIG. 16 shows the binding activity of antibodies TB-C2, and TB-D2 to B7H3 proteins.

FIG. 17 shows the two-terminal binding activity of antibodies TB-A1, TB-E, TB-B, and TB-F.

FIG. 18 shows the two-terminal binding activity of antibodies TB-G, TB-D1, and TB-H.

FIG. 19 shows the two-terminal binding activity of antibody TB-C1.

FIG. 20 shows the two-terminal binding activity of antibodies TB-A1, TB-D1, and TB-C1.

FIG. 21 shows the anti-tumor growth curves of antibodies TB-C1 and TB-C2.

FIG. 22 shows the binding activity of antibodies TD-A1, TD-E, TD-B, and TD-F to TNFR2 proteins.

FIG. 23 shows the binding activity of antibodies TD-C, TD-G, TD-D1, and TD-H to TNFR2 proteins.

FIG. 24 shows the binding activity of antibodies TD-A1, and TD-A2 to TNFR2 proteins.

FIG. 25 shows the binding activity of antibodies TD-D1, and TD-D2 to TNFR2 proteins.

FIG. 26 shows the ADCC activity of antibodies TD-A1 and TD-D1.

### DETAILED DESCRIPTION

The present invention is further described below in conjunction with the accompanying drawings and specific examples, and the protection content of the present invention is not limited to the following examples. It should also be understood that the terms used in the examples of the present invention are to describe specific embodiments, rather than to limit the scope of protection of the present invention. Without departing from the spirit and scope of the inventive concept, changes and advantages that can be thought of by those skilled in the art are all included in the present invention, and the attached claims and any equivalents thereof are the scope of protection of the present invention.

### Example 1 Preparation of Bispecific Antibodies with Different Structures

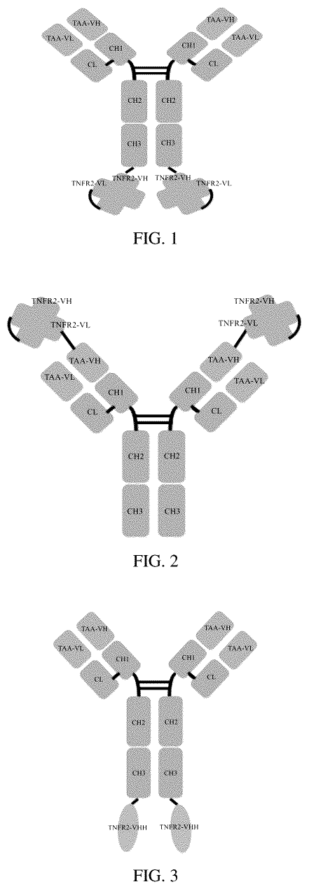

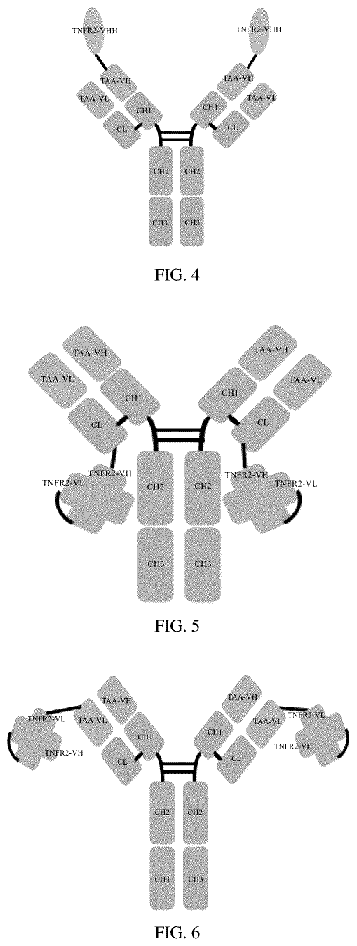

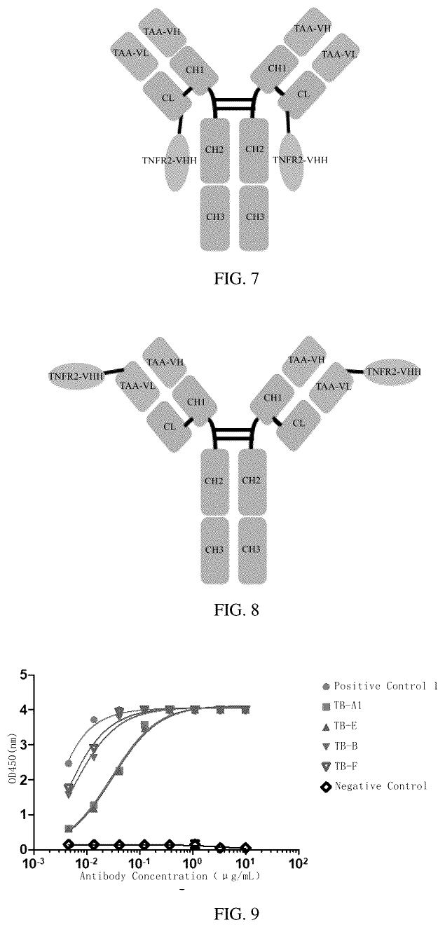

For TNFR2 and B7H3, bispecific antibodies were constructed according to the eight structures in FIGS. 1-8, respectively, and named TB-A to TB-H in order. For TNFR2 and CD24, bispecific antibodies were constructed according to the eight structures in FIGS. 1-8, respectively, and named TD-A to TD-H in order. For TNFR2 and Claudin18.2, bispecific antibodies were constructed according to the four structures in FIGS. 1, 2, 5 and 6, respectively, and named TC-A, TC-B, TC-E, and TC-F in order. The bispecific antibodies with part of structures of the present patent simultaneously construct IgG1 and IgG4 subtypes, and the antibody name followed by “1” is the IgG1 subtype, and the antibody name followed by “2” is the IgG4 subtype, for example: antibody TB-A1 is an antibody against TNFR2 and B7H3 targets, constructed according to the structure of FIG. 1 for the IgG1 subtype, and antibody TB-A2 is an antibody against TNFR2 and B7H3 targets, constructed according to the structure of FIG. 1 for the IgG4 subtype.

The sequences of anti-B7H3 antibody, anti-CD24 antibody and anti-Claudin18.2 antibody when used as full-length antibody and TNFR2 antibody when used as scFv or VHH are shown in Table 1.

TABLE 1
Sequence table

| | TAA | | | | TNFR2 | |
|---|---|---|---|---|---|---|
| | B7H3(Full- | CD24(Full- | Claudin18.2(Full- | Claudin 18.2(Full- | Sequence | Sequence |
| Name | length antibody) | length antibody) | length antibody) | length antibody) | 1 (scFv) | 2 (VHH) |
| VH | SEQ ID | SEQ ID | SEQ ID | SEQ ID | SEQ ID | SEQ ID |
| | NO: 1 | NO: 9 | NO: 17 | NO: 25 | NO: 33 | NO: 41 |
| HCDR1 | SEQ ID | SEQ ID | SEQ ID | SEQ ID | SEQ ID | SEQ ID |
| | NO: 2 | NO: 10 | NO: 18 | NO: 26 | NO: 34 | NO: 42 |
| HCDR2 | SEQ ID | SEQ ID | SEQ ID | SEQ ID | SEQ ID | SEQ ID |
| | NO: 3 | NO: 11 | NO: 19 | NO: 27 | NO: 35 | NO: 43 |
| HCDR3 | SEQ ID | SEQ ID | SEQ ID | SEQ ID | SEQ ID | SEQ ID |
| | NO: 4 | NO: 12 | NO: 20 | NO: 28 | NO: 36 | NO: 44 |
| VL | SEQ ID | SEQ ID | SEQ ID | SEQ ID | SEQ ID | / |
| | NO: 5 | NO: 13 | NO: 21 | NO: 29 | NO: 37 | |
| LCDR1 | SEQ ID | SEQ ID | SEQ ID | SEQ ID | SEQ ID | / |
| | NO: 6 | NO: 14 | NO: 22 | NO: 30 | NO: 38 | |
| LCDR2 | SEQ ID | SEQ ID | SEQ ID | SEQ ID | SEQ ID | / |
| | NO: 7 | NO: 15 | NO: 23 | NO: 31 | NO: 39 | |
| LCDR3 | SEQ ID | SEQ ID | SEQ ID | SEQ ID | SEQ ID | / |
| | NO: 8 | NO: 16 | NO: 24 | NO: 32 | NO: 40 | |

The sequences of TNFR2 antibody and the sequences of B7H3 antibody, CD24 antibody and Claudin18.2 antibody are linked by linkers in a certain order to form the peptide chains in Tables 2-4. Among them, in this embodiment, the linker used in the bispecific antibody has 2 GGGGS repeats (i.e., GGGGSGGGGS, hereinafter abbreviated as (G₄S)₂) or 3 GGGGS repeats (i.e., GGGGSGGGGSGGGGS, hereinafter abbreviated as (G₄S)₃), and the CL used in this embodiment is of the kappa (κ) type and the sequence is shown in SEQ ID NO: 45.

TABLE 2
Sequence list of peptide chains of bispecific antibodies (TNFR2 + B7H3)

| | | Amino acid | note |
|---|---|---|---|
| Name | Order of structure | sequence | |
| L1 | VL(B7H3)-CL | SEQ ID NO: | / |
| | | 46 | |
| L2 | VL(B7H3)-CL-(G4S)2-VH(TNFR2)-(G4S)3- | SEQ ID NO: | / |
| | VL(TNFR2) | 47 | |
| L3 | VL(B7H3)-CL-(G4S)2-VHH(TNFR2) | SEQ ID NO: | / |
| | | 48 | |
| L4 | VHH(TNFR2)-(G4S)3-VL(B7H3)-CL | SEQ ID NO: | / |
| | | 49 | |
| L5 | VH(TNFR2)-(G4S)3-VL(TNFR2)-(G4S)3-VL(B7H3)- | SEQ ID NO: | / |
| | CL | 50 | |
| H1 | VH(B7H3)-CH1-CH2-CH3-(G4S)3-VH(TNFR2)- | SEQ ID NO: | IgG1 subtype |
| | (G4S)3-VL(TNFR2) | 51 | |
| H2 | VH(B7H3)-CH1-CH2-CH3 | SEQ ID NO: | IgG1 subtype |
| | | 52 | |
| H3 | VH(TNFR2)-(G4S)3-VL(TNFR2)-(G4S)3- | SEQ ID NO: | IgG1 subtype |
| | VH(B7H3)-CH1-CH2-CH3 | 53 | |
| H4 | VH(B7H3)-CH1-CH2-CH3-(G4S)3-VHH(TNFR2) | SEQ ID NO: | IgG1 subtype |
| | | 54 | |
| H5 | VHH(TNFR2)-(G4S)3-VH(B7H3)-CH1-CH2-CH3 | SEQ ID NO: | IgG1 subtype |
| | | 55 | |
| H6 | VH(B7H3)-CH1-CH2-CH3-(G4S)3-VH(TNFR2)- | SEQ ID NO: | IgG4 subtype |
| | (G4S)3-VL(TNFR2) | 56 | |
| H7 | VH(B7H3)-CH1-CH2-CH3-(G4S)3-VHH(TNFR2) | SEQ ID NO: | IgG4 subtype |
| | | 57 | |
| H8 | VHH(TNFR2)-(G4S)3-VH(B7H3)-CH1-CH2-CH3 | SEQ ID NO: | IgG4 subtype |
| | | 58 | |

TABLE 3
Sequence list of peptide chains of bispecific antibodies (TNFR2 + CD24)

| | | Amino | |
|---|---|---|---|
| | | acid | |
| Name | Order of structure | sequence | note |
| L1 | VL(CD24)-CL | SEQ ID | / |
| | | NO: 59 | |
| L2 | VL(CD24)-CL-(G4S)2-VH(TNFR2)-(G4S)3-VL(TNFR2) | SEQ ID | / |
| | | NO: 60 | |
| L3 | VH(TNFR2)-(G4S)3-VL(TNFR2)-(G4S)3-VL(CD24)-CL | SEQ ID | / |
| | | NO: 61 | |
| L4 | VL(CD24)-CL-(G4S)2-VHH(TNFR2) | SEQ ID | / |
| | | NO: 62 | |
| L5 | VHH(TNFR2)-(G4S)3-VL(CD24)-CL | SEQ ID | / |
| | | NO: 63 | |
| H1 | VH(CD24)-CH1-CH2-CH3-(G4S)3-VH(TNFR2)-(G4S)3- | SEQ ID | IgG1 |
| | VL(TNFR2) | NO: 64 | subtype |
| H2 | VH(CD24)-CH1-CH2-CH3 | SEQ ID | IgG1 |
| | | NO: 65 | subtype |
| H3 | VH(TNFR2)-(G4S)3-VL(TNFR2)-(G4S)3-VH(CD24)- | SEQ ID | IgG1 |
| | CH1-CH2-CH3 | NO: 66 | subtype |
| H4 | VH(CD24)-CH1-CH2-CH3-(G4S)3-VHH(TNFR2) | SEQ ID | IgG1 |
| | | NO: 67 | subtype |
| H5 | VHH(TNFR2)-(G4S)3-VH(CD24)-CH1-CH2-CH3 | SEQ ID | IgG1 |
| | | NO: 68 | subtype |
| H6 | VH(CD24)-CH1-CH2-CH3-(G4S)3-VH(TNFR2)-(G4S)3- | SEQ ID | IgG4 |
| | VL(TNFR2) | NO: 69 | subtype |
| H7 | VHH(TNFR2)-(G4S)3-VH(CD24)-CH1-CH2-CH3 | SEQ ID | IgG4 |
| | | NO: 70 | subtype |

TABLE 4
Sequence list of peptide chains of bispecific antibodies
(TNFR2 + Claudin18.2)

| | | Amino acid |
|---|---|---|
| Name | Order of structure | sequence |
| L1 | VL(Claudin18.2)-CL | SEQ ID NO: |
| | | 71 |
| L2 | VL(Claudin18.2)-CL | SEQ ID NO: |
| | | 72 |
| L3 | VL(Claudin18.2)-CL-(G4S)2-VH(TNFR2)-(G4S)3-VL(TNFR2) | SEQ ID NO: |
| | | 73 |
| L4 | VL(Claudin18.2)-CL-(G4S)2-VH(TNFR2)-(G4S)3-VL(TNFR2) | SEQ ID NO: |
| | | 74 |
| L5 | VH(TNFR2)-(G4S)3-VL(TNFR2)-(G4S)3-VL(Claudin18.2)-CL | SEQ ID NO: |
| | | 75 |
| L6 | VH(TNFR2)-(G4S)3-VL(TNFR2)-(G4S)3-VL(Claudin18.2)-CL | SEQ ID NO: |
| | | 76 |
| H1 | VH(Claudin18.2)-CH1-CH2-CH3-(G4S)3-VH(TNFR2)-(G4S)3- | SEQ ID NO: |
| | VL(TNFR2) | 77 |
| H2 | VH(Claudin18.2)-CH1-CH2-CH3-(G4S)3-VH(TNFR2)-(G4S)3- | SEQ ID NO: |
| | VL(TNFR2) | 78 |
| H3 | VH(Claudin18.2)-CH1-CH2-CH3 | SEQ ID NO: |
| | | 79 |
| H4 | VH(Claudin18.2)-CH1-CH2-CH3 | SEQ ID NO: |
| | | 80 |
| H5 | VH(TNFR2)-(G4S)3-VL(TNFR2)-(G4S)3-VH(Claudin18.2)-CH1- | SEQ ID NO: |
| | CH2-CH3 | 81 |
| H6 | VH(TNFR2)-(G4S)3-VL(TNFR2)-(G4S)3-VH(Claudin18.2)-CH1- | SEQ ID NO: |
| | CH2-CH3 | 82 |

According to the combination of peptide chains in Tables 2-4, the amino acid sequences of the bispecific antibodies shown in Table 5 were designed and obtained.

TABLE 5
Bispecific antibody sequences

| | Amino acid sequence of | Amino acid sequence of |
|---|---|---|
| Antibody | the first peptide chain | the second peptide chain |
| TB-A1 | SEQ ID NO: 46 | SEQ ID NO: 51 |
| TB-A2 | SEQ ID NO: 46 | SEQ ID NO: 56 |
| TB-B | SEQ ID NO: 46 | SEQ ID NO: 53 |
| TB-C1 | SEQ ID NO: 46 | SEQ ID NO: 54 |
| TB-C2 | SEQ ID NO: 46 | SEQ ID NO: 57 |
| TB-D1 | SEQ ID NO: 46 | SEQ ID NO: 55 |
| TB-D2 | SEQ ID NO: 46 | SEQ ID NO: 58 |
| TB-E | SEQ ID NO: 47 | SEQ ID NO: 52 |
| TB-F | SEQ ID NO: 50 | SEQ ID NO: 52 |
| TB-G | SEQ ID NO: 48 | SEQ ID NO: 52 |
| TB-H | SEQ ID NO: 49 | SEQ ID NO: 52 |
| TD-A1 | SEQ ID NO: 59 | SEQ ID NO: 64 |
| TD-A2 | SEQ ID NO: 59 | SEQ ID NO: 69 |
| TD-B | SEQ ID NO: 59 | SEQ ID NO: 66 |
| TD-C | SEQ ID NO: 59 | SEQ ID NO: 67 |
| TD-D1 | SEQ ID NO: 59 | SEQ ID NO: 68 |
| TD-D2 | SEQ ID NO: 59 | SEQ ID NO: 70 |
| TD-E | SEQ ID NO: 60 | SEQ ID NO: 65 |
| TD-F | SEQ ID NO: 61 | SEQ ID NO: 65 |
| TD-G | SEQ ID NO: 62 | SEQ ID NO: 65 |
| TD-H | SEQ ID NO: 63 | SEQ ID NO: 65 |
| TC-A1 | SEQ ID NO: 71 | SEQ ID NO: 77 |
| TC-A2 | SEQ ID NO: 72 | SEQ ID NO: 78 |
| TC-B1 | SEQ ID NO: 71 | SEQ ID NO: 81 |
| TC-B2 | SEQ ID NO: 72 | SEQ ID NO: 82 |
| TC-E1 | SEQ ID NO: 73 | SEQ ID NO: 79 |
| TC-E2 | SEQ ID NO: 74 | SEQ ID NO: 80 |
| TC-F1 | SEQ ID NO: 75 | SEQ ID NO: 79 |
| TC-F2 | SEQ ID NO: 76 | SEQ ID NO: 80 |

The above-designed different structures of the bispecific antibody chains were genetically synthesized, and then the antibody fragments were inserted into PCDNA3.1 vector by using molecular cloning technology, constructed into mammalian cell expression plasmid, and then imported into the host cell line CHO cells by using liposome transfection, the fermentation supernatant was obtained by using cell Fed-batch, and the supernatant of the fermentation broth was purified by a series of steps such as affinity chromatography and ion exchange chromatography, and the constructed antibody was finally purified and obtained. The expression level, purity and SDS-PAGE of the purified antibody were detected to confirm the bispecific antibody.

### Example 2 Detection of the Binding Activity of Antibodies to TNFR2 Protein by ELISA (TNFR2+B7H3)

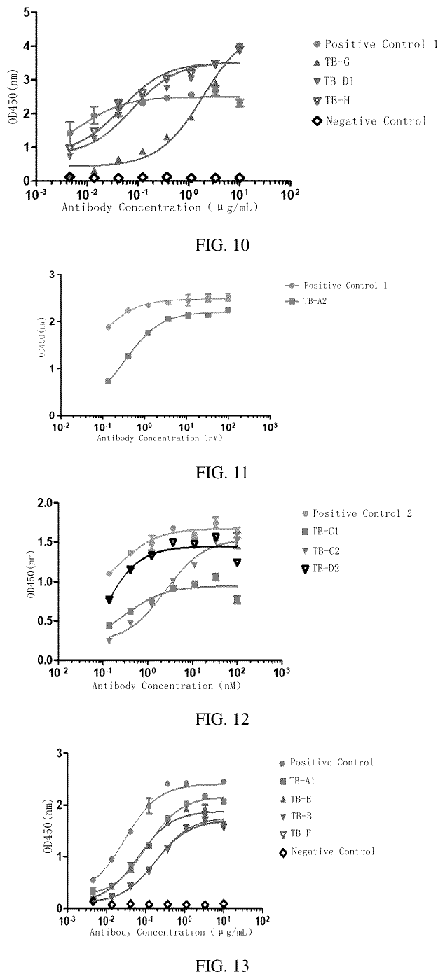

Human-TNFR2-His (manufacturer: SINO, CAT: 10417-H08H, LOT: LC15NO0412) was diluted to 0.3 μg/mL with a coating solution (1×PBS, pH7.4), and coated into a 96-well ELISA plate at 100 μL/well overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available trastuzumab for injection (Herceptin). The variable region of the antibody sequence of positive control 1 consisted of SEQ ID NO: 83 and SEQ ID NO: 84, and the constant region of human IgG1 was added (SEQ ID NO: 85 and SEQ ID NO: 86); the variable region of the antibody sequence of positive control 2 consisted of SEQ ID NO: 41. The positive control and antibody were diluted to 10 μg/mL with 3% skim milk powder as the starting concentration, and then diluted by 3-fold gradient (11 gradients in total). A blank well was set up and only the diluent was added. It was incubated at 37° C. for 1 h at 100 μL/well. The liquid in the well was discarded, washed 4 times with a plate washer, and patted dry on a flat paper. Goat anti-human IgG Fc was diluted with 3% skim milk powder at 1:20000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 9-12, respectively, and the results show that the antibodies TB-A1 to TB-H can bind to human TNFR2 protein.

### Example 3 Detection of the Binding Activity of Antibodies to B7H3 Protein by ELISA (TNFR2+B7H3)

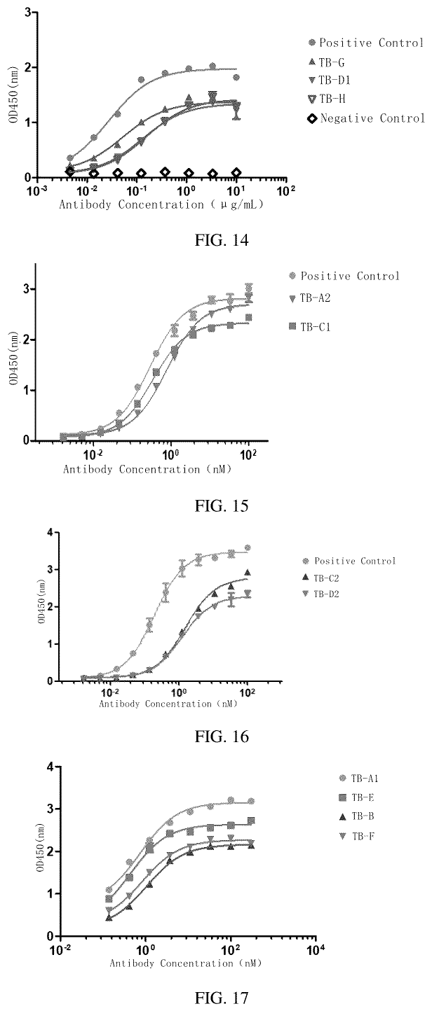

Human-B7H3 His (manufacturer: Acro, CAT: TB73-H52E2, LOT: 2052-21AXF1-YS) was diluted to 0.3 μg/mL with a coating solution (1×PBS, pH7.4), and coated into a 96-well ELISA plate at 100 μL/well overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available trastuzumab for injection (Herceptin). The variable region of the antibody sequence of the positive control consists of SEQ ID NO: 1 and SEQ ID NO: 5, with the constant region of human IgG1 added (SEQ ID NO: 85 and SEQ ID NO: 86). The positive control and antibody were diluted to 10 μg/mL with 3% skim milk powder as the starting concentration, and then diluted by 3-fold gradient (11 gradients in total). A blank well was set up and only the diluent was added. It was incubated at 37° C. for 1 h at 100 μL/well. The liquid in the well was discarded, washed 4 times with a plate washer, and patted dry on a flat paper. Goat anti-human IgG Fc was diluted with 3% skim milk powder at 1:20000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 13-16, respectively, and the results show that the antibodies TB-A1 to TB-H can bind to human B7H3 protein.

### Example 4 Dual Target Binding Activity of the Constructed Antibodies (TNFR2+B7H3)

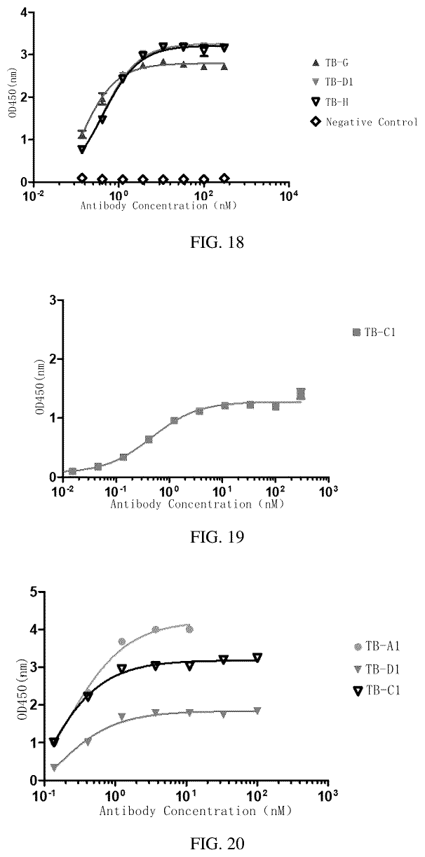

Human-B7H3 Fc (manufacturer: Acro, CAT: B73-H5253, LOT: 2053b-201XF1-UD) was diluted to 0.5 μg/mL with a coating solution (1×PBS, pH7.4) and coated into a 96-well ELISA plate at 100 μL/well at 4° C. overnight. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control was set up, which was commercially available trastuzumab for injection (Herceptin). Starting at 300 nM, which was used as the initial concentration for 3-fold dilution, a total of 8 gradients were diluted, and a blank well was set up, and only diluent was added. It was incubated at 37° C. for 1 h at 100 μL/well. The liquid in the well was discarded, washed 4 times with a plate washer, and patted dry on a flat paper. Human-TNFR2-His (manufacturer: Sino, CAT: 10417-H08H, LOT: LC12SE2902) was diluted to 0.5 μg/mL, 100 μL was added to each well, and incubated at room temperature for 1 h. The plate was washed 3 times with PBST, and then the HRP-labeled his antibody was diluted with sample diluent at 1:10000, 100 μL was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 17-19, respectively, and the results show that the antibodies TB-A1 to TB-H have binding activity at both ends.

### Example 5 Dual Target Binding Activity of the Constructed Antibodies (TNFR2+B7H3)

Human-TNFR2 mFc (manufacturer: Kaixia, CAT: TN-HM3R2, LOT: 031202) was diluted to 0.5 μg/mL with a coating solution (1×PBS, pH7.4) and coated into a 96-well ELISA plate at 100 μL/well at 4° C. overnight. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Starting at 100 nM, which was used as the initial concentration for 3-fold dilution, a total of 11 gradients were diluted, and a blank well was set up, and only diluent was added. It was incubated at 37° C. for 1 h at 100 L/well. The liquid in the well was discarded, washed 4 times with a plate washer, and patted dry on a flat paper. Human-TNFR2-His (Manufacturer: Acro, CAT: TB73-H52E2, LOT: 2052-21AXF1-YS) was diluted to 0.5 μg/mL, 100 μL was added to each well, and incubated at room temperature for 1 h. The plate was washed 3 times with PBST, and then the HRP-labeled his antibody was diluted with sample diluent at 1:10000, 100 μL was added to each well, and incubated at room temperature for 1 h. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIG. 20, and the results show that the antibodies TB-A1, TB-D1 and TB-C1 have binding activity at both ends.

### Example 6 Assay of CD8+ T Cell Activation (TNFR2+B7H3)

PBS diluted antibody to 70 nM (experimental final concentration 35 nM, 50 μL/well); PBS diluted OKT3 to 10 μg/mL (experimental final concentration 5 μg/mL, 50 μL/well). 50 μL of OKT3 dilution and 50 μL of antibody dilution, irrelevant antibody was set up, and the irrelevant antibody was human IgG1 kappa Isotype control (HG1K, Sino Biological), negative control was set up, and it was coated into a 96-well flat-bottomed plates with a total volume of 100 μL/well, and incubated at 4° C. overnight. On the next day, CD8+ T cells were negatively isolated from frozen PBMC (ORICELLS, ID:PCH20210300033, 30% isolation efficiency), and the sorted CD8+ T cells were tested for purity; CFSE labeled the CD8+ T cells at a concentration of 5 μM, and the staining was terminated by the addition of complete medium after incubation at 37° C. for 10 min. The plate used for coating as described before was washed once with PBS, and CFSE-labeled or unlabeled T cells were added at 2×10⁵/well, with 100 μL/well, and Anti-CD28 at a final concentration of 1 μg/mL, with 100 μL/well. The total system of the experiment was 200 μL/well. 96 h of co-incubation was performed at 37° C.; the supernatant was collected after centrifugation and used for the detection of IFNγ and IL2 cytokines, and the cells were washed with PBS to perform the flow-through assay. The results are shown in Table 6, the agonistic antibodies TB-A1, TB-B, TB-E, and TB-F all show activation effects on CD8+ T cells.

TABLE 6
Results of CD8+ T cell activation assay

| groups | Proliferation (%) | | | mean value |
|---|---|---|---|---|
| CD8+ T cell | 0.89 | 0.84 | 0.73 | 0.82 |
| Unactivated T-cells | 4.29 | 10.38 | 6.15 | 6.94 |
| (CFSE) | | | | |
| Negative control | untested | 67.37 | 65.22 | 66.30 |
| TB-A1 | 78.63 | 79.11 | 78.58 | 78.77 |
| TB-E | 78.3 | 76.39 | 79.17 | 77.95 |
| TB-B | 77.04 | 78.07 | 77.79 | 77.63 |
| TB-F | 77.12 | 79.45 | 79.21 | 78.59 |
| Irrelevant antibody | 68.16 | 67.28 | 63.58 | 66.34 |

### Example 7 Detection of the Affinity of Antibodies to TNFR2 Protein by BLI Method (TNFR2+B7H3)

The AHC sensor was soaked in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibody in the sample plate was solidified for 300 s, and the secondary equilibration buffer was used for 180 s. 100 nM human-TNFR2-His (manufacturer: SINO, CAT: 10417-H08H, LOT: LC12SE2902) protein was bound to the antibody for 300 s and then dissociated for 600 s. After dissociation, 10 mM glycine (pH 2.0) was used as a regeneration buffer for 30 s. The sensor was regenerated with 10 mM glycine (pH 2.0). The affinity results of the antibody molecules are shown in Table 7. The results show that the affinity of the TNFR2 end of the antibodies TB-A1, TB-C1, and TB-D1 were all at the nanomolar level or sub-nanomolar level.

TABLE 7
Affinity of antibody for TNFR2 protein

| Solidified material | KD(M) | Kon(1/Ms) | Kdis(1/s) |
|---|---|---|---|
| TB-A1 | 2.34 × 10−10 | 2.23 × 10⁵ | 5.22 × 10−5 |
| TB-C1 | 2.89 × 10−9 | 2.30 × 10⁵ | 6.65 × 10−4 |
| TB-D1 | 1.35 × 10−9 | 3.94 × 10⁵ | 5.31 × 10−4 |

### Example 8 Detection of the Affinity of Antibodies to B7H3 Protein by BLI Method (TNFR2+B7H3)

The AHC sensor was soaked in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibody in the sample plate was solidified for 300 s, and the secondary equilibration buffer was used for 180 s. 100 nM human-TNFR2-His (Manufacturer: Acro, CAT: TB73-H52E2, LOT: 2052-21AXF1-YS) protein was bound to the antibody for 300 s and then dissociated for 600 s. After dissociation, 10 mM glycine (pH 2.0) was used as a regeneration buffer for 30 s. The sensor was regenerated with 10 mM glycine (pH 2.0). The affinity results of the antibody molecules are shown in Table 8. The results show that the antibodies TB-A1, TB-C1 and TB-D1 all have affinity for the B7H3 protein.

TABLE 8
Affinity of antibodies to B7H3 protein

| Solidified material | KD(M) | Kon(1/Ms) | Kdis(1/s) |
|---|---|---|---|
| TB-A1 | 2.20 × 10−8 | 4.07 × 10⁴ | 8.94 × 10−4 |
| TB-C1 | 1.59 × 10−7 | 1.09 × 10⁴ | 1.74 × 10−3 |
| TB-D1 | 9.45 × 10−9 | 3.30 × 10⁴ | 3.11 × 10−4 |

### Example 9 Pharmacodynamic Study of Each Test Substance in the Subcutaneously Transplanted Mouse Colon Cancer Cell Line MC38-hB7H3 Tumor Model in hTNFR2 Mice (TNFR2+B7H3)

Female C57BL/6-hTNFR2 mice at 6-7 weeks of age were selected and subcutaneously inoculated with MC38-hB7H3 tumors, respectively, and randomly divided into four groups when the tumor volume was about 100±50 mm3. There were five mouse in each group, and the subgroups consisted of (1) G1: PBS group; (2) G2: control antibody group, the sequence variable region consisted of SEQ ID NO: 83 and SEQ ID NO: 84, joined with the constant region of human IgG1 (see SEQ ID NO: 85 and SEQ ID NO: 86); (3) G3: antibody TB-C1 group; and (4) G4: antibody TB-C2 group. The negative control group was administered intratumorally using PBS, and the remaining group samples were administered intratumorally at 10 mg/kg. The frequency of administration was 2 times per week for 4 consecutive weeks for a total of 8 times; the tumor volume and body weight of the mouse were measured every other day, and the tumor volume was calculated, according to a-b²/2 (a is the long diameter, b is the short diameter). The experimental protocol design is shown in Table 9.

TABLE 9
Experimental protocol design

| | | | | | | Frequency of | |
|---|---|---|---|---|---|---|---|
| | | | | | | administration | total |
| | | | Investigational | Dose | route of | and | sample |
| Group | Mouse | Cell | drug | (mg/kg) | administration | observation | size |
| G1 | 5 | 5 × 10⁵ | / | / | intraperitoneal | 2 times per | / |
| | | | | | injection | week | |
| G2 | 5 | 5 × 10⁵ | Control | 10 | intraperitoneal | 2 times per | 8~10 mg |
| | | | antibody | | injection | week | |
| G3 | 5 | 5 × 10⁵ | Antibody | 10 | intraperitoneal | 2 times per | 8~10 mg |
| | | | TB-C1 | | injection | week | |
| G4 | 5 | 5 × 10⁵ | Antibody | 10 | intraperitoneal | 2 times per | 8~10 mg |
| | | | TB-C2 | | injection | week | |

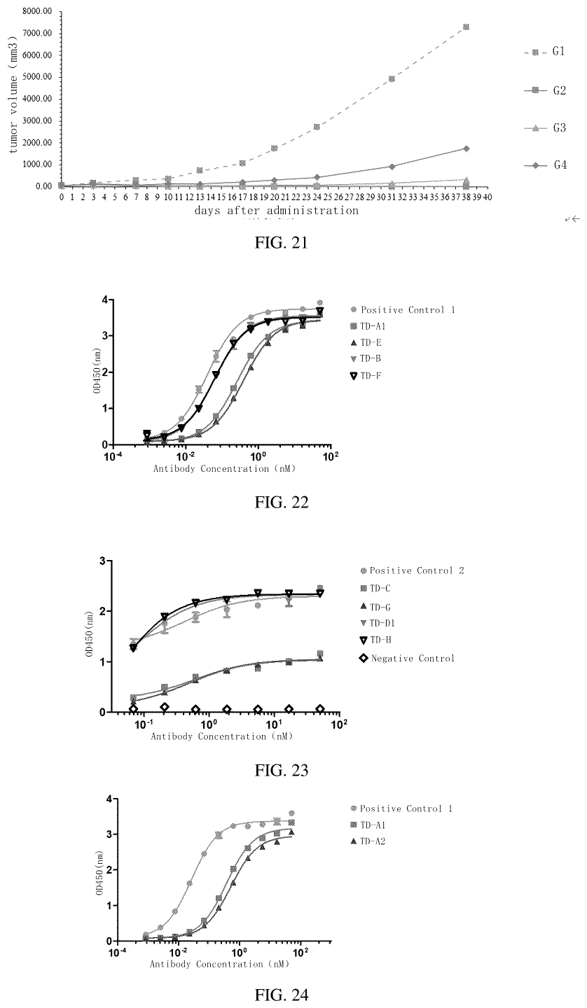

The results are shown in FIG. 21, and it can be seen from the tumor growth curve that antibody TB-C1 and antibody TB-C2 have obvious anti-tumor effects.

### Example 10 Detection of the Binding Activity of Antibodies to TNFR2 Protein by ELISA (TNFR2+CD24)

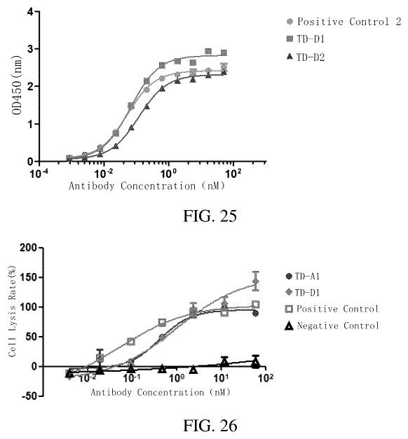

Human-TNFR2-His (manufacturer: SINO, CAT: 10417-H08H, LOT: LC12SE2902) was diluted to 0.3 μg/mL with a coating solution (1×PBS, pH7.4), and coated into a 96-well ELISA plate at 100 μL/well overnight at 4° C. The coating solution was poured off. Each well was washed with 1×PBST at 300 μL, washed 4 times with a plate washer, and patted dry on a flat paper. It was blocked with 3% skim milk powder at 300 μL/well, and incubated at 37° C. for 1 h. The blocking solution was poured off, washed 4 times with a plate washer, and patted dry on a flat paper. Negative control and positive control were set up. The negative control was commercially available trastuzumab for injection (Herceptin). The variable region of the antibody sequence of positive control 1 consisted of SEQ ID NO: 83 and SEQ ID NO: 84, and the constant region of human IgG1 was added (see SEQ ID NO: 85 and SEQ ID NO: 86); the variable region of the antibody sequence of positive control 2 consisted of SEQ ID NO: 41. The positive control and antibody were diluted to 10 μg/mL with 3% skim milk powder as the starting concentration, and then diluted by 3-fold gradient (11 gradients in total). A blank well was set up and only the diluent was added. It was incubated at 37° C. for 1 h at 100 μL/well. The liquid in the well was discarded, washed 4 times with a plate washer, and patted dry on a flat paper. Goat anti-human IgG Fc was diluted with 3% skim milk powder at 1:20000, and incubated at 37° C. for 1 h at 100 μL/well. It was washed 6 times with a plate washer, and patted dry on a flat paper. TMB color developing solution was added at 100 μL/well, and wrapped with aluminum foil. Color development was performed at 37° C. in the dark for 8 min. Termination solution 1M HCl was added to terminate the color development reaction at 100 μL/well. Readings were taken at 450 nm on an ELISA reader. The ELISA results of the antibody molecules are shown in FIGS. 22-25, respectively, and the results show that the antibodies TD-A1 to TD-H can bind to human TNFR2 protein.

### Example 11 Detection of the Binding Activity of Antibodies to CD24 Protein by Flow Cytometry (TNFR2+CD24)

A positive control was set up, the variable region of antibody sequence of the positive control consisted of SEQ ID NO: 9 and SEQ ID NO: 13, and the constant region of human IgG1 (see SEQ ID NO: 85 and SEQ ID NO: 86) was joined, and dilution was performed, and the actual final concentration of the positive control and the antibody to be tested was set 150 nM at 100 μL per well. Tumor cells MCF-7 (purchased from CAS Cell Bank, catalog number: TCHu74) highly expressing human CD24 were washed twice, 1×10⁵/well, and added into 96-well plates at 100 μL/tube. Positive control and antibody to be tested were added into the 96-well plate sequentially with 50 μL/well, and equal volumes of dilutions were added to the blank control group and negative control group. After mixing, the 96-well plate was incubated at 4° C. for 1 h. Pre-cooled dilutions were added and washed once, centrifuged (3000 rpm, 3 min), the supernatant was discarded, and the cells were harvested. Fluorescent secondary antibody goat anti-human IgG Fc (manufacturer Biolegend, Cat. No.: 398004, Lot No.: B340957) was taken and diluted at the used concentration of 1:500. 100 μL of dilution was added to the blank control group, and 100 μL of fluorescent secondary antibody dilution was added to the rest of the sample groups respectively. After incubation for 0.5 h at room temperature and 4° C. protected from light, PBS was added to wash and centrifuged (3000 rpm, 3 min) twice. After discarding the supernatant, 80 μL of the dilution was added and resuspended. The cells in each group were sequentially flow assayed, and the results of antibody molecules were shown in Table 10, which shows that the antibodies TD-A1 to TD-H can bind to MCF-7 tumor cells with high expression of human CD24.

TABLE 10
Binding activity of antibodies TD-A1 to TD-H to CD24 protein

| Antibody name | E1 Mean PE-A |
|---|---|
| Positive control | 4454716 |
| TD-H | 4136909 |
| TD-D1 | 4028752 |
| TD-B | 4024844 |
| TD-G | 4024449 |
| TD-F | 3721344 |
| TD-E | 3300474 |
| TD-A1 | 3279104 |
| TD-C | 3149715 |
| Negative control | 6066 |
| Blank control | 5600 |

### Example 12 Binding Activity at Both Ends of the Constructed Antibodies (TNFR2+CD24)

Negative control group was set up, negative control 1 (PBS group), negative control 2 had a variable region of antibody sequence consisting of SEQ ID NO: 83 and SEQ ID NO: 84, which was joined to the constant region of human IgG1 (see SEQ ID NO: 85 and SEQ ID NO: 86); negative control 3 had a variable region of antibody sequence consisting of SEQ ID NO: 9 and SEQ ID NO: 13, which was joined to the constant region of human IgG1 (see SEQ ID NO: 85 and SEQ ID NO: 86). Dilution was performed, and the actual final concentration of the negative control and antibody to be tested was set 150 nM, and 100 μL per well. Tumor cells MCF-7 (purchased from the CAS Cell Bank, Catalog No. TCHu74) highly expressing human CD24 were washed twice, 1×10⁵/well, and added into the 96-well plate at 100 μL/tube. Negative control and antibody to be tested were sequentially added into 96-well plates with 50 μL/well, and equal volumes of dilutions were added to the blank control group and negative control group. Human TNFR2 protein (SINO, CAT: 10417-H08H) was sequentially added into 96-well plate with 50 μL/well, and after mixing, the 96-well plate was incubated at 4° C. for 1 h. Pre-cooled dilution was added and washed once, centrifuged (3000 rpm, 3 min), and the supernatant was discarded, and the cells were harvested. Fluorescent secondary antibody Anti-6X-his tag antibody (FITC) (manufacturer: abcam, Cat. No.: ab1206, Lot No.: GR3420599-3) was taken and diluted at the used concentration of 1:500. 100 μL of dilution was added to the blank control group, and 100 μL of fluorescent secondary antibody dilution was added to the rest of the sample groups respectively. After incubation for 0.5 h at room temperature and 4° C. protected from light, PBS was added to wash and centrifuged (3000 rpm, 3 min) twice. After discarding the supernatant, 80 μL of the dilution was added and resuspended. The cells in each group were sequentially flow assayed, and the results of antibody molecules are shown in Table 11, which show that the antibodies TD-A1 to TD-H can bind both TNFR2 protein and MCF-7 tumor cells with high expression of human CD24.

TABLE 11
Binding activity at both ends of the antibodies

| Antibody name | E1 Mean FITC-A |
|---|---|
| TD-A1 | 7227393 |
| TD-C | 7200647 |
| TD-G | 6674700 |
| TD-D1 | 6316316 |
| TD-E | 5594786 |
| TD-B | 5234019 |
| TD-H | 3119992 |
| TD-F | 2606235 |
| Negative control 3 (CD24) | 58605 |
| Blank control | 35736 |
| Negative control 2 | 33314 |
| (TNFR2) | |
| Negative control 1 (PBS) | 33186 |

### Example 13 Detection of the Affinity of Antibodies to TNFR2 Protein by BLI Method (TNFR2+CD24)

Positive controls were set up, the variable region of the antibody sequence of positive control 1 consists of SEQ ID NO: 83 and SEQ ID NO: 84, and the constant region of human IgG1 was added (SEQ ID NO: 85 and SEQ ID NO: 86); the variable region of the antibody sequence of positive control 2 consists of SEQ ID NO: 41. The AHC sensor was soaked in 0.02% PBST (0.02% Tween 20, pH 7.4, 1×PBS) as a buffer for 600 s to remove the sucrose covering the sensor surface. The AHC sensor was equilibrated with 0.02% PBST (0.02% Tween 20, pH 7.4, 1×PBS) as a buffer for 60 s, the antibody in the sample plate was solidified for 300 s, and the secondary equilibration buffer was used for 180 s. 100 nM human-TNFR2-His (manufacturer: SINO, CAT: 10417-H08H, LOT: LC12SE2902) protein was bound to the antibody for 300 s and then dissociated for 600 s. After dissociation, 10 mM glycine (pH 2.0) was used as a regeneration buffer for 30 s. The sensor was regenerated with 10 mM glycine (pH 2.0). The affinity results of the antibody molecules are shown in Table 12. The results show that the affinity of the TNFR2 end of the antibodies TD-A1, TD-C, TD-D1, TD-G and TD-H were all at the nanomolar level or sub-nanomolar level.

TABLE 12
Affinity of antibodies to TNFR2 protein

| Name | KD(M) | kon(1/Ms) | kdis(1/s) |
|---|---|---|---|
| Positive control 1 | 3.24 × 10−10 | 3.91 × 10⁵ | 1.27 × 10−4 |
| TD-A1 | 5.23 × 10−10 | 2.36 × 10⁵ | 1.23 × 10−4 |
| Positive control 2 | 1.61 × 10−9 | 2.66 × 10⁵ | 4.27 × 10−4 |
| TD-C | 2.38 × 10−9 | 2.75 × 10⁵ | 6.52 × 10−4 |
| TD-G | 2.54 × 10−9 | 1.51 × 10⁵ | 3.83 × 10−4 |
| TD-D1 | 1.44 × 10−9 | 4.64 × 10⁵ | 6.67 × 10−4 |
| TD-H | 1.28 × 10−9 | 4.94 × 10⁵ | 6.34 × 10−4 |

### Example 14 Assay of CD8+ T Cell Activation (TNFR2+CD24)

PBS diluted antibody to 70 nM (experimental final concentration 35 nM, 50 μL/well); PBS diluted OKT3 to 10 μg/mL (experimental final concentration 5 μg/mL, 50 μL/well). Irrelevant antibody was set up, the irrelevant antibody was human IgG1 kappa Isotype control (HG1K, Sino Biological), negative control and positive control groups were set up, and the variable region of the antibody sequence of the positive control consisted of SEQ ID NO: 83 and SEQ ID NO: 84, which was joined to the constant region of human IgG1 (SEQ ID NO:85 and SEQ ID NO: 86). 50 μL of OKT3 dilution and 50 μL of antibody dilution, and it was coated into a 96-well flat-bottomed plates with a total volume of 100 μL/well, and incubated at 4° C. overnight. On the next day, CD8+ T cells were negatively isolated from frozen PBMC (ORiCELLS, ID: PCH20210300033, 30% isolation efficiency), and the sorted CD8+ T cells were tested for purity; CFSE labeled the CD8+ T cells at a concentration of 5 μM, and the staining was terminated by the addition of complete medium after incubation at 37° C. for 10 min. The plate used for coating as described before was washed once with PBS, and CFSE-labeled or unlabeled T cells were added at 2×10⁵/well, with 100 μL/well, and Anti-CD28 at a final concentration of 1 μg/mL, with 100 μL/well. The total system of the experiment was 200 μL/well. 96 h of co-incubation was performed at 37° C.; the supernatant was collected after centrifugation and used for the detection of IFNγ and IL2 cytokines, and the cells were washed with PBS to perform the flow-through assay. The results are shown in Table 13, the agonistic antibody TD-A1 shows activation effects on CD8+ T cells.

TABLE 13
Results of CD8+ T cell activation assay

| Group | Proliferation (%) | | | mean value |
|---|---|---|---|---|
| CD8+ T cell | 1.32 | 1.98 | 0.65 | 1.32 |
| Unactivated T-cells (CFSE) | 0.28 | 0.27 | 0.47 | 0.34 |
| Negative control | 60.95 | 61.45 | 61.87 | 61.42 |
| Positive control | 78.63 | 79.11 | 78.58 | 78.77 |
| TD-A1 | 78.3 | 76.39 | 79.17 | 77.95 |
| Irrelevant antibody | 71.21 | 71.11 | 70.36 | 70.89 |

### Example 15 ADCC Activity of Antibodies Against MCF-7 Cells (TNFR2+CD24)

Negative control (IgG1 isotype control) and positive control (variable region of the antibody sequence consisting of SEQ ID NO: 9 and SEQ ID NO: 13, with a constant region of human IgG1 joined, see SEQ ID NO: 85 and SEQ ID NO: 86) were set up. MCF-7 cells overexpressing human CD24 (purchased from the CAS Cell Bank, catalog no. TCHu74) were used as target cells, and after centrifugation at 1000 rpm for 4 min at room temperature and resuspension using RPMI1640 basal medium (containing 5% FBS), they were spread in 96-well plates at 1×10⁴ cells/well, and 50 μL/well; The antibody was diluted using RPMI1640 basal medium (containing 5% FBS) at a initial concentration of 120 nM, while a 5-fold gradient of dilutions with 8 concentration gradients of 100 μL/well was followed; NK158V cells were resuspended and added to the corresponding wells at 50 μL/well, and E: T was 3:1. target cell maximum lysis wells (M), target cell spontaneous release wells (ST), effector cell spontaneous release wells (SE), total volume-corrected blank wells (BV), and medium blank control wells (BM) were also set up. After standing for 10 min, centrifuge at 1000 rpm for 4 min at room temperature and incubate in a 5% CO₂, 37° C. CO₂ cell culture incubator for 4 h. 20 μL lysate was added to wells M and B-V 45 min ahead of time, mixed well, and centrifuged at 1000 rpm for 4 min at room temperature at the end of incubation. 50 μL of supernatant was aspirated to the LDH analysis plate, then 50 μL/well of substrate dissolved in analysis buffer (assay buffer) was added, and the reaction was carried out for 30 min at room temperature and protected from light, then 50 μL/well of termination solution was added, and it was left to stand for 10 min, and readings were taken at 490 nm to calculate the cell death rate.

$$Cell Death Rate ( \% ) = \frac{{ODvalue}_{sample well - S T - S E} \times 100}{{ODvalue}_{M - ST}}$$

The concentration of the antibody was logarithmically taken as the horizontal coordinate, and the Sigmoidaldose-response (VariableSlope) method (GraphPadPrism Software, GraphPadSoftware, SanDiego, California) was chosen to perform a nonlinear regression to obtain the target antibody activity on the ADCC activity profile of the target cells.

As can be seen in FIG. 26, the negative control does not show killing of MCF-7 cells, and the positive control, antibody TD-A1 and antibody TD-D1 show significant cleavage death of MCF-7 cells in a concentration-dependent manner.

### Example 16 Binding Activity at Both Ends of the Constructed Antibodies (TNFR2+Claudin18.2)

Negative control group, negative control 2 (antibody sequence consists of SEQ ID NO: 87 and SEQ ID NO: 88), negative control 3 (antibody sequence consists of SEQ ID NO: 79 and SEQ ID NO: 71), irrelevant antibody (human IgG1 kappa Isotype control, manufacturer: Sino Biological, Cat. No.: HG1K, Lot No.: MA140C2803) were set up. Negative control, irrelevant antibody and antibody to be tested were diluted and the actual final concentration was set 150 nM, with 100 μL per well. Tumor cells CHOK1-18.2 (P6-4-1) (purchased from the CAS Cell Bank, catalog number: TCHu74) highly expressing human Claudin18.2 were washed twice, 1×10⁵/well, and added into the 96-well plate at 100 μL/tube. Negative control 2, negative control 3, irrelevant antibody and antibody to be tested were sequentially added into 96-well plates with 50 μL/well, and equal volumes of dilutions were added to the blank control group and negative control group. Human TNFR2 protein (SINO, CAT: 10417-H08H) was sequentially added into 96-well plate with 100 μL/well, and after mixing, the 96-well plate was incubated at 4° C. for 1 h. Pre-cooled dilution was added and washed once, centrifuged (3000 rpm, 3 min), and the supernatant was discarded, and the cells were harvested. Fluorescent secondary antibody Anti-6X-his tag antibody (FITC) (manufacturer: abcam, Cat. No.: ab1206, Lot No.: GR3420599-3) was taken and diluted at the used concentration of 1:500. 100 μL of dilution was added to the blank control group, and 100 μL of fluorescent secondary antibody dilution was added to the rest of the sample groups respectively. After incubation for 0.5 h at room temperature and 4° C. protected from light, PBS was added to wash and centrifuged (3000 rpm, 3 min) twice. After discarding the supernatant, 80 μL of the dilution was added and resuspended. The cells in each group were sequentially flow assayed, and the results of antibody molecules are shown in Table 14, which shows that all antibodies can bind both TNFR2 protein and CHOK1-18.2 tumor cells with high expression of human Claudin18.2.

TABLE 14
Binding activity at both ends of the antibodies

| Antibody name | E1 Mean FITC-A |
|---|---|
| TC-A1 | 280611 |
| TC-E1 | 275421 |
| TC-E2 | 260075 |
| TC-F2 | 247861 |
| TC-A2 | 216622 |
| TC-F1 | 141287 |
| TC-B2 | 139293 |
| TC-B1 | 55204 |
| Blank control | 13476 |
| Negative control 1(PBS) | 17780 |
| Negative control 2(TNFR2) | 16286 |
| Negative control 3(Claudin18.2) | 15698 |
| IgG1(Human IgG1 kappa Isotype control, | 14372 |
| Manufacturer: Sino Biological, Cat. | |
| No.: HG1K, Lot No.: MA140C2803) | |

The protection scope of the present invention is not limited to the above embodiments. Without departing from the spirit and scope of the inventive conception, variations and advantages that can be thought of by those skilled in the art are included in the present invention and are protected by the appended claims.

1. A bispecific antibody, comprising: (a) a first antibody or antigen-binding fragment thereof that specifically binds to a first antigen; and (b) a second antibody or antigen-binding fragment thereof that specifically binds to a second antigen; wherein the first antigen is a tumor-associated antigen (TAA), and the second antigen is TNFR2.

2. The bispecific antibody of claim 1, wherein the first antibody or antigen-binding fragment thereof comprises a heavy chain and a light chain; and the second antibody or antigen-binding fragment thereof comprises scFv or VHH.

3. The bispecific antibody of claim 2, wherein the heavy chain variable region of one heavy chain and the light chain variable region of one light chain of the first antibody form an antigen-binding site, and the heavy chain variable region of another heavy chain and the light chain variable region of another light chain form an antigen-binding site.

4. The bispecific antibody of claim 3, further comprising one first antibody or antigen-binding fragment thereof and one or more scFv or VHH.

5. The bispecific antibody of claim 4, further comprising one first antibody or antigen-binding fragment thereof and one scFv or one VHH, wherein the scFv or VHH is connected to the N-terminus or C-terminus of the heavy chain of the first antibody or the antigen-binding fragment thereof.

6. The bispecific antibody of claim 4, further comprising one first antibody or antigen-binding fragment thereof and two scFvs or two VHHs.

7. The bispecific antibody of claim 6, wherein the two scFvs or two VHHs are respectively connected to the N-terminus or C-terminus of two heavy chains of the first antibody or antigen-binding fragment thereof.

8. The bispecific antibody of claim 7, further comprising two first polypeptide chains and two second polypeptide chains, wherein for each of the polypeptide chains: (a) the first polypeptide chain independently comprises the light chain of the first antibody or antigen-binding fragment thereof; and (b) the second polypeptide chain independently comprises the heavy chain of the first antibody or antigen-binding fragment thereof and the scFv or VHH.

9. The bispecific antibody of claim 8, wherein the two first polypeptide chains are identical or different, and/or the two second polypeptide chains are identical or different.

10. The bispecific antibody of claim 9, wherein the tumor-associated antigen (TAA) is selected from the group consisting of: GPC3, CD19, CD20 (MS4A1), CD22, CD24, CD30, CD33, CD38, CD40, CD123, CD133, CD138, CDK4, CEA, Claudin18.2, AFP, ALK, B7H3, BAGE protein, BCMA, BIRC5 (survivin), BIRC7, β-catenin (β-catenin), brc-ab1, BRCA1, BORIS, CA9, CA125, carbonic anhydrase IX, cysteinyl asparagin-8 (caspase-8), CALR, CCR5, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, GM3, gp100, Her2, HLA/B-raf, HLA/k-ras, HLA/MAGE-A3, hTERT, IL13Rα2, LMP2, κ-Light, LeY, MAGE-1, MAGE-2, MAGE-3, MAGE-4, MAGE-6, MAGE-12, MART-1, mesothelin, ML-IAP, MOv-γ, Muc1, Muc2, Muc3, Muc4, Muc5, Muc16, MUM1, Ras, RGS5, Rho, ROR1, SART-1, SART-3, STEAP1, STEAP2, TAG-72, TGF-β, TMPRSS2, Tang-Noble Antigen, TRP-1, TRP-2, tyrosinase and urolysin-3, 5T4.

11. The bispecific antibody of claim 10, wherein the tumor-associated antigen (TAA) is selected from the group consisting of: B7H3, CD24, or Claudin18.2.

12. The bispecific antibody of claim 11, wherein the heavy chain variable region and the light chain variable region of the scFv are connected by a linker L1.

13. The bispecific antibody of claim 12, wherein the scFv or VHH is connected to the N-terminus or C-terminus of the heavy chain of the first antibody or antigen-binding fragment thereof by a linker L2.

14. The bispecific antibody of claim 13, wherein the linker L1 and linker L2 are identical or different.

15. The bispecific antibody of claim 14, wherein the linker L1 and/or linker L2 has an amino acid sequence as shown in (G₄S)ₓ, where x is an integer selected from 1-6.

16. The bispecific antibody of claim 15, wherein the heavy chain of the first antibody or antigen-binding fragment thereof comprises a heavy chain variable region and a heavy chain constant region, and the light chain comprises a light chain variable region and a light chain constant region.

17. The bispecific antibody of claim 16, wherein the heavy chain of the first antibody or antigen-binding fragment thereof comprises a first Fc region and a second Fc region.

18. The bispecific antibody of claim 17, wherein the first Fc region and the second Fc region are identical or different.

19. The bispecific antibody of claim 18, wherein the first Fc region or the second Fc region is selected from IgG, IgA, IgD, IgE, IgM, or variants thereof.

20. The bispecific antibody of claim 19, wherein the first Fc region or the second Fc region is selected from IgG1, IgG2, IgG3, IgG4, or variants thereof.

21-51. (canceled)
